(19) United States
(12) Patent Application Publication
(10) Pub. No.: US 2026/0335455 A1
(43) Pub. Date: Oct. 29, 2026

(54) DRUG CONJUGATE TARGETING CTLA4, PRODUCTS COMPRISING THE SAME AND THERAPEUTIC USES THEREOF

(71) Applicants: INSTITUT GUSTAVE ROUSSY, VILLEJUIF (FR); UNIVERSITE PARIS-SACLAY, GIF SUR YVETTE (FR); INSTITUT NATIONAL DE LA SANTE ET DE LA RECHERCHE MEDICALE, PARIS (FR)

(72) Inventors: SANDRINE SUSINI, PARIS (FR); LAMBROS TSELIKAS, CHARENTON LE PONT (FR); AURÉLIEN MARABELLE, SCEAUX (FR); ROMANE MARTINEAU, SAINT VINCENT DES LANDES (FR)

(21) Appl. No.: 19/491,149

(22) PCT Filed: Jun. 7, 2024
(86) PCT No.: PCT/EP2024/065790

§ 371 Date: Dec. 8, 2025

(30) Foreign Application Priority Data
Jun. 9, 2023 (EP) 23305926.0

(51) Int. Cl. A61K 47/68; A61K 39/00; A61P 35/00; C07K 16/28

(52) U.S. Cl. CPC A61K 47/68031; A61K 47/68033; A61K 47/6849; A61K 47/6851; A61K 47/6889; A61P 35/00; C07K 16/2818; A61K 2039/505

(57) ABSTRACT

The present invention relates to a new drug conjugate comprising a CTLA4 targeting molecule, at least one cytotoxic agent, and a linker connecting the CTLA4 targeting molecule and the cytotoxic agent, as well as to compositions and kits comprising such a drug conjugate, and to uses thereof in particular for treating cancer and/or for preventing a cancer relapse in a subject in need thereof.

### FIELD OF THE INVENTION

The present invention relates to a new drug conjugate comprising a Cytotoxic-T-Lymphocyte-Antigen 4 protein (CTLA4) targeting molecule, preferably an anti-CTLA4 monoclonal antibody, at least one cytotoxic agent, and a linker connecting the CTLA4 targeting molecule and the cytotoxic agent, as well as combinations, compositions and kits comprising such a drug conjugate, and to uses thereof in particular for treating cancer and/or for preventing a cancer relapse in a subject in need thereof.

### BACKGROUND OF THE INVENTION

Following the therapeutic success observed in melanoma and lung cancer (Antonia et al., 2017; Eggermont et al., 2015, 2016, 2018, 2020; Gao et al., 2020; Luke et al., 2022), anti-PD(L) 1 and anti-CTLA4 immunotherapies are currently being developed for primary (localized) tumors in neo-adjuvant and adjuvant settings, as well as in metastatic settings for most cancers. However, gains in relapse-free survival and overall survival for localized and metastatic cancers are achieved at the cost of severe adverse autoimmune or inflammatory toxic events of grade 3 to 5 according to Common Terminology Criteria for Adverse events (CTCAE), events also known as immune related adverse events (or irAEs). This toxicity is observed in a higher proportion of patients suffering of a localized cancerous tumor than in metastatic patients (>30%).

The mechanism of action of anti-CTLA4 therapy relies on CTLA4 immune checkpoint blockade (antagonistic to CD80 and CD86) and on the depletion of CTLA4+ lymphocytes induced by Antibody Derived Cell Cytotoxicity (ADCC) or Antibody Derived Cell Phagocytosis (ADCP). Two anti-CTLA4 monoclonal antibodies are currently FDA and EMA approved: ipilimumab (IgG1; Yervoy®, Bristol Myers Squibb) and tremelimumab (IgG2; Imjudo®, Astra Zeneca). Intravenous anti-CTLA4 immunotherapy is currently approved for the treatment of localized melanoma and metastatic Melanoma (“MM”), Renal Cell Carcinoma (RCC), MSI-H/MMRd (Micro Satellite Instable High/Mismatch Repair Deficient) colorectal Cancer, Hepatocellular Carcinoma, Non-Small Cell Lung Cancer (“NSCLC”) and Malignant Pleural Mesothelioma, but at the cost of significant irAEs.

With regard to the treatment of cancers involving the intravenous administration of products, an insufficient antitumor effect of those products is typically related to the limitation of the dose that can be administered systemically because of on-target off-tumor adverse events. Indeed, the approved doses and regimens of these products is typically determined in Phase I clinical trials with a dose escalation part that stop at the maximum tolerated doses when treated patients faced dose limiting toxicities. Also, the combination of immunotherapies are limited by their additional and sometime synergistic effects on systemic toxicities. Last but not least, the treatment of local cancers (Stages I-II, +/−III) with systemic immunotherapies is limited by the level of irreversible and sometime fatal effects of immunotherapies administered intravenously where the benefit/risk ratio is not comparable to the one observed for patients suffering of advanced relapsing/refractory cancers.

Thus, it has remained an objective in oncology to achieve a superior therapeutic effect while further enhancing safety of cancer treatments.

Inventors herein provide new therapeutic tools advantageously usable in the context of cancer treatment.

### SUMMARY OF THE INVENTION

Inventors have developed and now herein advantageously describe for the first time a CTLA4 targeting drug conjugate. This drug conjugate comprises i) a Cytotoxic-T-Lymphocyte-Antigen 4 protein (CTLA4) targeting molecule, preferably an anti-CTLA4 monoclonal antibody, ii) at least one cytotoxic agent, and iii) a linker connecting the CTLA4 targeting molecule and the cytotoxic agent. A particular herein described drug conjugate is an anti-CTLA4 Antibody-Drug Conjugate (“ADC”).

In a particular aspect, the anti-CTLA4 ADC comprises i) ipilimumab or tremelimumab as the anti-CTLA4 monoclonal antibody, ii) any cytotoxic agent as herein described, and iii) any linker as herein described connecting the anti-CTLA4 monoclonal antibody and the cytotoxic agent. Patent application WO2020/092155 describes a particular polypeptide having a heavy chain variable region and/or light chain variable region that specifically binds to CTLA4 protein as well as antibodies and fragments containing the same. Byrne et al. describes a “CTLA-4-targeted engineered toxin body (ETB)” (“MT-8421”) which may be used alone or after a treatment with a αPD-1 monoclonal antibody. However, none of these documents describe or suggest a drug conjugate according to the present invention comprising an anti-CTLA4 monoclonal antibody (mAb) such as ipilimumab or tremelimumab. On the contrary, when describing combination therapy, they drive the skilled person away from anti-CTLA-4 mAbs, described as responsible for adverse events, and recommend a more selective approach involving the use of very specific anti-CTLA4 polypeptides.

The herein described CTLA4 targeting drug conjugate allows an advantageously superior therapeutic effect compared to the effect observed with a treatment involving the (intravenous or intra-tumoral) administration of the sole anti-CTLA4 agent, with much reduced toxicity.

In a particular aspect, the cancerous subject to be treated is a subject whose tumor or tumor Micro Environment (TME) comprises CTLA4+ cells, preferably CTLA4+ tumor cells and/or CTLA4+ immune cells.

In another particular aspect, the cancerous subject is a subject having (primary or secondary) resistance to (naked, i.e., unmodified, uncoupled or unlinked to any other agent) anti-CTLA4 and/or anti-PD(L)1 agent(s), in particular a subject whose tumor and/or TME displays no, low level of, or dysfunctional myeloid cells, such as macrophages, said myeloid cells expressing no or low levels of the Fcγ receptor I (“CD64”), Fcγ receptor IIIa (“CD16a”) and/or Fcγ receptor IIIb (“CD16b”).

For example, the subject to be treated is a subject whose tumor or TME does not comprise myeloid cells, in particular macrophages, positive for the Fcγ receptor I (“CD64”), Fcγ receptor IIIa (“CD16a”) and/or Fcγ receptor IIIb (“CD16b”), or a subject who comprises a proportion of myeloid cells, in particular macrophages, positive for the Fcγ receptor I (“CD64”), Fcγ receptor IIIa (“CD16a”) and/or Fcγ receptor IIIb (“CD16b”) inferior to the proportion detected in a cancerous patient, or population of cancerous patients, suffering from the same cancer identified as exhibiting a durable clinical benefit (DCB) for an anti-cancer treatment, preferably an anti-cancer treatment, involving in particular an anti-CTLA-4 monoclonal antibody such as for example ipilimumab or tremelimumab [used alone or in combination with an additional conventional anti-cancer therapeutic agent such as an anti-PD1 (for example nivolumab)].

Inventors also herein describe a pharmaceutical combination involving, or pharmaceutical composition comprising, at least one drug conjugate of the invention, for example at least two distinct drug conjugates, a first conjugate comprising a first cytotoxic agent and a second conjugate comprising a distinct cytotoxic agent, the composition comprising in addition a pharmaceutically acceptable support, excipient, carrier, or diluent. They also describe a pharmaceutical combination involving, or pharmaceutical composition comprising, at least one drug conjugate of the invention, and distinct therapeutic agent(s) for example selected from an immune checkpoint targeting agent, an anti-angiogenic agent, a cytotoxic agent, an hormonal agent and any combination thereof, the composition comprising in addition a pharmaceutically acceptable support, excipient, carrier, or diluent.

A kit is also described. This kit comprises at least two drug conjugates of the invention, or at least one drug conjugate of the invention, and distinct therapeutic agent(s) for example selected from an immune checkpoint targeting agent, an anti-angiogenic agent, a cytotoxic agent, an hormonal agent and any combination thereof.

Further herein described are the drug conjugate, combination, composition and kit herein disclosed for the first time for use for treating a cancer or for preventing a cancer relapse in a subject in need thereof, as well as any method for preventing or treating cancer comprising a step of administering the drug conjugate, combination or composition comprising the same to the subject in need thereof.

### DETAILED DESCRIPTION OF THE INVENTION

Unless defined otherwise, all technical and scientific terms used herein have the same meaning as commonly understood by one of ordinary skill in the art to which the subject-matter disclosed herein belongs.

The following definitions may be useful to understand embodiments as presented herein.

Unless otherwise indicated, the terms “cancer”, “cancerous tumor”, “malignant tumor”, “tumor”, “neoplasia”, “cancer disease”, “malignancy”, or “proliferative disease”, are herein used interchangeably. These terms refer to or describe the physiological condition in mammals that is typically characterized by unregulated cell proliferation, possibly associated with tumor formation and growth and/or systemic dissemination of proliferating cells (metastases). As used herein “cancer” refers to any malignant and/or invasive growth or tumor caused by abnormal cell proliferation. As used herein “cancer” refers to solid tumors named for the type of cells that form them, as well as cancer of blood, bone marrow, or the lymphatic system. Examples of solid tumors include but are not limited to melanomas, sarcomas and carcinomas. Examples of cancers of the blood include but are not limited to leukemias, lymphomas and myeloma. The term “cancer” includes but is not limited to a primary cancer that originates at a specific site in the body. The term cancer also includes a cancer that has metastasized, i.e., that has spread from the place in which it started to other parts of the body, for example to the brain, bone, lung, or liver; a recurrence from the original primary cancer after remission, and a second primary cancer that is a new primary cancer in a person with a history of previous cancer of a different type from latter one.

In a particular aspect, the cancer is characterized by malignant tumor and/or metastasis present in the brain, bone, lung, or liver.

In the context of the invention, a “tumor cell” or a “cancer cell” is a cell obtained from a tumor or tissue of a subject suffering from a cancer or at risk of developing a cancer, in particular from at least one of the herein identified cancers, for example melanoma, sarcoma or carcinoma, and exhibiting well-known hallmarks of cancer cells, e.g. sustaining proliferative signaling, evading growth suppressors, resisting cell death, enabling replicative immortality, inducing angiogenesis, and activating invasion and metastasis. It is to be understood that the expression “tumor cells” used to identify cells obtained from a tumor of a subject, is also used, in the present description, to identify circulating tumor cells, cells obtained from a liquid tumor biopsy, cells obtained from a tumor bed, or cells obtained from a metastasis. The term “tumor cell” designates, in addition to cancerous cells, any cell present in the tumor such as for example a stromal cell (for example a fibroblast or a vessel cell) or an immune cell.

In a particular aspect, the tumor is a malignant (cancerous) tumor. In another particular aspect, the tumor is a “pre-malignant” (precancerous) tumor.

In the context of the present invention, a “conventional treatment of cancer” (also herein identified as “standard-of-care treatment” or “main mode of cancer therapy”) refer to the therapy routinely applied or, if not routinely applied, appropriate and at least recommended by health authorities. The “conventional” treatment is chosen by the oncologist depending on the specific cancer to be prevented or treated. The conventional cancer treatment may involve for example a cytotoxic agent, an anti-angiogenic agent, an anti-hormone agent (hormonotherapy), an immunotherapeutic agent (immunotherapy), and/or the exposition of the tumor to radiations (radiotherapy).

The term “adjuvant therapy” refers to additional treatment given after a main mode of therapy, notably after the surgical resection of a primary tumor.

The term “neoadjuvant” and “neoadjuvant setting” refer to a treatment performed before surgery.

In recent years, scientific and clinical advances have improved the understanding of the role and phenotype of T-regulatory lymphocytes (Tregs) in the context of cancer immune tolerance.

Inventors identified the phenotype of tumor antigen-specific Tregs among other tumor-infiltrating lymphocytes. They showed that their presence within the tumor microenvironment (“TME”) is critical to the efficacy of immunotherapies targeting the CTLA4 and PD-1 immune checkpoints. Inventors in particular identified that membrane CTLA-4 is highly expressed by these tumour antigen-specific Tregs, and showed that, upon fixation to an anti-CTLA-4 agent such as ipilimumab, CTLA-4 is internalized into the cytoplasm of CTLA-4+ cells together with said anti-CTLA-4 agent.

Based on their work, Inventors herein provide novel therapeutic compounds, combinations of compounds, and pharmaceutical compositions comprising such compounds, and describe uses thereof in oncology. They more particularly developed and herein describe for the first time a CTLA4 targeting drug conjugate (also herein identified simply as “the drug conjugate”) which is significantly more cytotoxic towards CTLA-4 expressing cells than the sole anti-CTLA-4 and far less toxic for the patient since the effective concentrations are for example more than 6 times lower than the standard concentration used for ipilimumab. Thus, the conjugate of the invention allows an advantageously superior benefice over risk therapeutic effect-if compared to the effect observed with a treatment involving the (systemic or intra-tumoral) administration of the sole anti-CTLA4 agent. Inventors in particular herein show that a CTLA4 targeting drug conjugate is advantageously capable of inducing the selective destruction of particular CTLA4+ population of cells, thereby allowing a more efficient treatment of cancer while simultaneously decreasing the toxicity of said treatment for the subject.

### the Drug Conjugate

Inventors herein describe for the first time a drug conjugate comprising i) a Cytotoxic-T-Lymphocyte-Antigen 4 protein (CTLA4) targeting molecule, preferably an anti-CTLA-4 monoclonal antibody, ii) at least one anti-cancer agent, preferably at least one cytotoxic agent, and iii) a linker connecting the CTLA4 targeting molecule and the cytotoxic agent, is herein described.

### The CTLA4 Targeting Molecule

The CTLA-4 or CTLA4 (cytotoxic T-lymphocyte-associated protein 4), also known as CD152 (cluster of differentiation 152), is a protein receptor that functions as an immune checkpoint and downregulates immune responses. CTLA-4 is constitutively expressed in regulatory T-cells (Treg) but only upregulated in conventional T cells after activation—a phenomenon which is particularly notable in cancers. CTLA4 acts as an “off” switch when bound to CD80 or CD86 on the surface of antigen-presenting cells. The CTLA4 protein is encoded by the Ctla-4 gene in mice (Brunet J F, Denizot F, Luciani MF, Roux-Dosseto M, Suzan M, Mattei M G, Golstein P (1987). “A new member of the immunoglobulin superfamily—CTLA4”. Nature. 328(6127): 267-70) and the CTLA-4 gene in humans (Dariavach P, Mattéi M G, Golstein P, Lefranc M P (December 1988). “Human Ig superfamily CTLA-4 gene: chromosomal localization and ientity of protein sequence between murine and human CTLA-4 cytoplasmic domains”. European Journal of Immunology. 18 (12): 1901-5.

CTLA-4 is a member of the immunoglobulin superfamily that is expressed by activated T cells and transmits an inhibitory signal to T cells. The CTLA-4 receptor downregulates the immune system. CTLA-4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA-4 transmits an inhibitory signal to T cells, whereas CD28 transmits a stimulatory signal. T cell activation through the T cell receptor and CD28 leads to increased expression of CTLA-4. CTLA-4 is found in particular as a receptor expressed at the membrane level by regulatory T cells (Tregs) after engagement of their TCR receptor (Rowshanravan et al., 2018), and contributes to their inhibitory function. The mechanism by which CTLA-4 acts in T cells remains somewhat controversial.

The CTLA4 protein contains an extracellular domain, a transmembrane domain, and a cytoplasmic tail. Alternate splice variants, encoding different isoforms, have been characterized. The membrane-bound isoform functions as a homodimer interconnected by a disulfide, while the soluble isoform functions as a monomer. The intracellular domain is similar to that of CD28, in that it has no intrinsic catalytic activity and contains one YVKM motif able to bind PI3K, PP2A and SHP-2 and one proline-rich motif able to bind SH3 containing proteins. The first role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 can also bind PI3K, although the importance and results of this interaction are uncertain.

In the context of the present invention, the CTLA4 targeting molecule is a molecule capable of recognizing and binding CTLA4 at the surface of a cell before being internalized by the CTLA4+ cell into the cytoplasm, for example of recognizing the CTLA4 extracellular domain. The CTLA4+ cell is for example a tumor cell or a lymphocyte, in particular an activated T lymphocyte, even more particularly a regulatory T cell (Treg).

The CTLA4 targeting molecule is preferably an anti-CTLA-4 monoclonal antibody such as for example ipilimumab or tremelimumab.

Another herein described CTLA4 targeting molecule is a molecule that can target and bind to, or in other words, that is directed against CTLA4.

The CTLA4 targeting molecule can be selected for example from a molecule capable of modulating, preferably capable of inhibiting or reducing, either directly or indirectly, the function of T cells, in particular the transmission of the herein above described inhibitory signal to T cells.

A particular CTLA4 targeting molecule is capable of selectively destroying CTLA4+ cells, in particular CTLA4+ cancer cells or CTLA4+ lymphocytes, preferably CTLA4+ Tregs, even more preferably CTLA4+ tumor-antigen specific Tregs (“tumor Tregs”).

Another particular CTLA4 targeting molecule is capable of mimicking or amplifying the biological function that CTLA4 exerts on Tregs. A particular and preferred CTLA4 targeting molecule is an anti-CTLA4 molecule inducing the depletion of Tregs in a subject, preferably the depletion of tumor-antigen specific Tregs (“tumor Tregs”), even more preferably the specific depletion of tumor Tregs and of Tregs present in the tumor microenvironment (TME) to the exclusion of other tumor infiltrating lymphocytes or of any other lymphocytes present in the subject.

In a particular aspect, the CTLA4 targeting molecule is an anti-CTLA4 antibody (immunoglobulin), any (functional) fragment thereof (including a single chain antibody) or any (functional) variant thereof which would be considered as equivalent by the skilled person. In the context of the present invention, the term “antibody” (or “immunoglobulin”) designates any kind of antibody such as a monoclonal antibody, a multispecific antibody (i.e. an antibody comprising a first antigen binding site and at least one second different antigen binding site; e.g. a bispecific antibody) or a single chain antibody. This term also covers any (functional) variant or (functional) fragment thereof.

A typical antibody consists of a glycoprotein comprising at least two heavy (H) chains and two light (L) chains inter-connected by disulfide bonds. Each heavy chain comprises a heavy chain variable region (or domain) (abbreviated herein as VH) and a heavy chain constant region (hereafter CH). Heavy chains are classified as gamma, mu, alpha, delta, or epsilon, and define the antibody's isotype as IgG, IgM, IgA, IgD, and IgE, respectively. The heavy chain constant region of the immunoglobulin IgG, IgD, and IgA (γ, δ and α chains respectively) comprises three domains (CH1, CH2, and CH3) and a hinge region for added flexibility, and the heavy chain constant region of the immunoglobulin IgM and IgE contains 4 domains (CH1, CH2, CH3, and CH4). The antibody of the invention can be of the IgG, IgM, IgA, IgD, and IgE isotype, depending on the structure of its heavy chain. However, in a preferred embodiment, the antibody of the invention is of the IgG isotype, i.e., its heavy chain is of the gamma (γ) type.

IgG antibodies are classified in four distinct subtypes, namely IgG1, IgG2, IgG3 and IgG4 in the order of their abundance in serum (IgG1 being the most abundant). The structure of the hinge regions in the γ chain gives each of these subtypes its unique biological profile (even though there is about 95% similarity between their Fc regions, the structure of the hinge regions is relatively different). The antibody of the invention can be of the IgG1, IgG2, IgG3 or IgG4 subtype. However, in a preferred embodiment, the antibody of the invention is of the IgG1 or IgG2 subtype.

Each light chain comprises a light chain variable region (abbreviated herein as VL) and a light chain constant region comprising only one domain, CL. There are two types of light chain in mammals: the kappa (κ) chain, encoded by the immunoglobulin kappa locus on chromosome 2, and the lambda (λ) chain, encoded by the immunoglobulin lambda locus on chromosome 22. In a preferred embodiment, the antibody of the invention has a Kappa light chain. The VH and VL regions can be further subdivided into regions of hypervariability, termed “Complementarity Determining Regions” (CDR), which are primarily responsible for binding an epitope of an antigen, and which are interspersed with regions that are more conserved, termed “Framework Regions” (FR). Each VH and VL is composed of three CDRs and four FRs, arranged from amino-terminus to carboxy-terminus in the following order: FR1, CDR1, FR2, CDR2, FR3, CDR3, FR4. The functional ability of the antibody to bind a particular antigen depends on the variable regions of each light/heavy chain pair, and is largely determined by the CDRs.

The variable region of the heavy chain differs in antibodies produced by different B cells, but is the same for all antibodies produced by a single B cell or B cell clone (or hybridome). By contrast, the constant regions of the antibodies mediate the binding of the immunoglobulin to host tissues or factors, including various cells of the immune system (e.g. effector cells) and the first component (Clq) of the classical complement system.

As used herein, the term “antibody fragments” intends to designate Fab, Fab′, F(ab′)2, scFv, dsFv, ds-scFv, single chain antibody, dimers, minibodies, nanobodies, diabodies, and multimers thereof and bispecific antibody fragments. Antibodies can be fragmented using conventional techniques. Various techniques have been developed for the production of antibody fragments. Traditionally, these fragments were derived via proteolytic digestion of intact antibodies. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, nanobodies, diabodies, bispecific antibody fragments and other fragments can also be synthesized by recombinant techniques.

Typically, the antibody fragment of the invention is a functional fragment, i.e. an antibody fragment capable of binding and preferably inhibiting or neutralizing the activity of a molecule of interest as does the antibody it is deriving from.

In a particular and preferred embodiment, the antibody of the invention is a monoclonal antibody. A “monoclonal antibody”, as used herein, designates an antibody arising from a nearly homogeneous population of antibodies. More particularly, the antibodies of a given subject are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one isotype and subtype, and light chains of only one type. In addition, in contrast with preparations of polyclonal antibodies, each monoclonal antibody is directed to a single epitope of an antigen.

To produce monoclonal antibodies, antibody producing cells (lymphocytes) can be harvested from an immunized animal as described above and fused with myeloma cells by standard somatic cell fusion procedures thereby immortalizing these cells and yielding hybridoma cells. Such techniques are well known in the art (e.g. the hybridoma technique originally developed by Kohler and Milstein (1975)) as well as other techniques such as the human B-cell hybridoma technique, the EBV-hybridoma technique to produce human monoclonal antibodies, and screening of combinatorial antibody libraries. Hybridoma cells can be screened immunochemically for production of antibodies specifically reactive with the target polypeptide(s) so that only monoclonal antibodies binding to said polypeptide(s) are isolated. The antibody or a fragment thereof of the invention may be a human, chimeric, humanized, murine, CDR-grafted, phage-displayed, bacteria-displayed, yeast-displayed, transgenic-mouse produced, mutagenized, or randomized antibody or fragment.

A chimeric antibody is a molecule in which different portions are derived from different animal species, such as those having a variable region derived from a murine monoclonal antibody (mAb) and a human immunoglobulin constant region.

Humanized forms of antibodies of the invention are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin (recipient antibody) are replaced by corresponding non-human residues of the donor antibody. Furthermore, humanized antibodies may comprise residues that are not found in the recipient antibody or in the donor antibody. In general, the humanized antibody may comprise substantially all of at least one, typically two, variable domains, in which all or substantially all of the hypervariable loops correspond to those of a non-human immunoglobulin (donor antibody having the desired specificity, affinity, and capacity) and all or substantially all of the FRs are those of a human immunoglobulin sequence. Methods for humanizing non-human antibodies have been described in the art. Preferably, a humanized antibody has one or more amino acid residues introduced into it from a source, which is non-human. These non-human amino acid residues are often referred to as “import” residues, which are typically taken from an “import” variable domain. Humanization may be essentially performed by substituting hypervariable region sequences for the corresponding sequences of a human antibody. Accordingly, such humanized antibodies are chimeric wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. Other methods generally involve conferring donor CDR binding affinity onto an antibody acceptor variable region framework. One method involves simultaneously grafting and optimizing the binding affinity of a variable region binding fragment. Another method relates to optimizing the binding affinity of an antibody variable region.

The antibody or fragment thereof of the invention may have other agents conjugated to them, such as drug, toxin or radioactive atom.

In a particular aspect, the CTLA4 targeting molecule is a DARPIN (designed ankyrin repeat protein), such as the MP0250 DARPin® drug, MP0317 DARPin® drug, or MP0533 DARPin® drug.

In another particular aspect, the CTLA4 targeting molecule is an aptamer, in particular an oligonucleotide DNA or RNA sequence binding specifically CTLA4, such as pegaptanib.

The CTLA4 targeting molecule is preferably an anti-CTLA4 antibody, even more preferably an anti-CTLA4 monoclonal antibody, for example quavonlimab, ipilimumab or tremelimumab, preferably ipilimumab or tremelimumab, even more preferably ipilimumab.

Ipilimumab is a monoclonal antibody that works to activate the immune system by targeting CTLA-4. Cytotoxic lymphocytes (CTLs) can recognize and destroy cancer cells. However, an inhibitory mechanism interrupts this destruction. Ipilimumab turns off this inhibitory mechanism and boosts the body's immune response against cancer cells (i.e., allows the lymphocytes to continue to destroy cancer cells). Ipilimumab was approved by the US Food and Drug Administration (FDA) in March 2011, for the treatment of melanoma. It is also approved in combination with Nivolumab for the treatment of advanced renal cell carcinoma, microsatellite instability (MSI-H) or mismatch repair deficient (dMMR) metastatic colorectal cancer, hepatocellular carcinoma, non-small cell lung cancer (NSCLC) and malignant pleural mesothelioma.

A major drawback of ipilimumab therapy is its association with severe and potentially fatal immunological adverse effects due to T cell activation and proliferation, occurring in ten to twenty percent of patients. Serious adverse effects include stomach pain, bloating, constipation, diarrhea, fever, trouble breathing, and urinating problems. Between 5.7 and 9.1% of individuals treated with ipilimumab develop checkpoint inhibitor induced colitis. Individual cases of severe neurologic disorders following ipilimumab have been observed, including acute inflammatory demyelination polyneuropathy and an ascending motor paralysis, as well as myasthenia gravis. Tremelimumab is a fully human monoclonal antibody used for the treatment of hepatocellular carcinoma (a type of liver cancer), designed to attach to and block CTLA-4. The most common side effects when used in combination with durvalumab include rash, pruritus (itching), diarrhea, abdominal (belly) pain, increased levels of liver enzymes, fever, hypothyroidism (an underactive thyroid gland), cough, peripheral edema (swelling especially of the ankles and feet) and increased level of lipase (an enzyme that helps digest fat, mainly made in the pancreas). Tremelimumab blocks the binding of the antigen-presenting cell ligands B7.1 and B7.2 to CTLA-4, resulting in inhibition of B7-CTLA-4-mediated downregulation of T cell activation. Subsequently, B7.1 or B7.2 may interact with another T-cell surface receptor protein, CD28, resulting in a B7-CD28-mediated T-cell activation unopposed by B7-CTLA-4-mediated inhibition. Unlike ipilimumab, which is an IgG1 isotype, tremelimumab is an IgG2 isotype.

Thus, a particular drug conjugate herein described is an antibody-drug conjugate (“ADC”) or a pharmaceutically acceptable polymorph, enantiomer, stereoisomer, salt, solvate or tautomer thereof. ADCs are a class of biopharmaceutical drugs designed as a targeted therapy for treating cancer. Unlike chemotherapy, ADCs are intended to target and kill tumor cells while sparing healthy cells. ADCs are complex molecules composed of an antibody linked to a biologically active payload, typically a cytotoxic (anti-cancer) agent or drug.

In the context of the present invention, the ADC comprises preferably a monoclonal anti-CTLA4 antibody such as ipilimumab or tremelimumab, preferably ipilimumab.

### Payload of the Drug Conjugate

Many of the payloads for oncology drug conjugate, typically ADCs, are natural product based. In a typical aspect, the payload is an antineoplastic agent, in particular a chemotherapeutic agent, for example a small molecule.

Payloads include for example the microtubule inhibitors such as for example monomethyl auristatin (MMAE), monomethyl auristatin F (MMAF) or a maytansinoid (such as DM1 or DM4); the DNA damaging agents such as for example a calicheamicin; the topoisomerase 1 inhibitors such as for example SN38 (active metabolite of irinotecan), or exatecan. Alternatives to small molecule payloads have also been investigated, for example, siRNA.

In a preferred aspect, the payload is an anticancer agent, preferably a cytotoxic agent, typically a small molecule.

The cytotoxic agent can be selected for example from a microtubule inhibitor (MTI), a DNA damaging agent such as an alkylating agent or a platinum complex; a cytotoxic antibiotic; an antimetabolite; a topoisomerase I inhibitor; a RNA Polymerase Inhibitor; an antimitotic agent, and any combination thereof.

The microtubule inhibitor (MTI) can be for example a taxane (such as for example paclitaxel or docetaxel); a vinca alkaloid (such as for example vinblastine (VBL), vinorelbine (VRL), vincristine (VCR) or vindesine (VDS)); or an epothilone (such as for example epotilone A, epothilone B, Ixabepilone, or a semi synthetic analog of epothilone B, epothilone C, epothilone D, epothilone E or epothilone F).

In a particular aspect, the microtubule inhibitor is selected from a Maytansinoid (such as DM1 or DM4), auristatin and a derivative thereof such as monomethyl auristatin E (MMAE) or monomethyl auristatin F (MMAF).

MMAE and MMAF are synthetic antineoplastic agents inhibiting cell division by blocking the polymerization of tubulin. Because of their high toxicity MMAE and MMAF cannot be used as a single-agent chemotherapeutic drug.

The auristatin derivative is preferably MMAF.

A particular conjugate comprises i) ipilimumab, ii) a microtubulin inhibitor as a cytotoxic agent, and iii) a cleavable linker comprising a valine-citrulline dipeptide or a phenylalanine-lysine dipeptide.

A particular conjugate comprises i) tremelimumab, ii) a microtubulin inhibitor as a cytotoxic agent, and iii) a cleavable linker comprising a valine-citrulline dipeptide or a phenylalanine-lysine dipeptide.

A particular conjugate comprises i) ipilimumab, ii) a maytansinoid as a cytotoxic agent, and iii) a cleavable linker comprising a valine-citrulline dipeptide or a phenylalanine-lysine dipeptide.

A particular conjugate comprises i) tremelimumab, ii) a maytansinoid as a cytotoxic agent, and iii) a cleavable linker comprising a valine-citrulline dipeptide or a phenylalanine-lysine dipeptide. A particular and preferred conjugate comprises i) ipilimumab, ii) DM1 as a cytotoxic agent, and iii) a cleavable linker comprising a valine-citrulline dipeptide or a phenylalanine-lysine dipeptide.

A particular and preferred conjugate comprises i) tremelimumab, ii) DM1 as a cytotoxic agent, and iii) a cleavable linker comprising a valine-citrulline dipeptide or a phenylalanine-lysine dipeptide.

A particular and preferred conjugate comprises i) ipilimumab, ii) DM4 as a cytotoxic agent, and iii) a cleavable linker comprising a valine-citrulline dipeptide or a phenylalanine-lysine dipeptide.

A particular and preferred conjugate comprises i) tremelimumab, ii) DM4 as a cytotoxic agent, and iii) a cleavable linker comprising a valine-citrulline dipeptide or a phenylalanine-lysine dipeptide.

A particular conjugate comprises i) ipilimumab, ii) auristatin or a derivative of auristatin as a cytotoxic agent, and iii) a cleavable linker comprising a valine-citrulline dipeptide or a phenylalanine-lysine dipeptide.

A particular conjugate comprises i) tremelimumab, ii) auristatin or a derivative or auristatin as a cytotoxic agent, and iii) a cleavable linker comprising a valine-citrulline dipeptide or a phenylalanine-lysine dipeptide.

A particular conjugate comprises i) ipilimumab, ii) DNA damaging agent as a cytotoxic agent, and iii) a cleavable linker comprising a valine-citrulline dipeptide or a phenylalanine-lysine dipeptide.

A particular conjugate comprises i) tremelimumab, ii) DNA damaging agent as a cytotoxic agent, and iii) a cleavable linker comprising a valine-citrulline dipeptide or a phenylalanine-lysine dipeptide.

A particular conjugate comprises i) ipilimumab, ii) calicheamicin as a cytotoxic agent, and iii) a cleavable linker comprising a valine-citrulline dipeptide or a phenylalanine-lysine dipeptide.

A particular conjugate comprises i) tremelimumab, ii) calicheamicin as a cytotoxic agent, and iii) a cleavable linker comprising a valine-citrulline dipeptide or a phenylalanine-lysine dipeptide.

A particular and preferred conjugate comprises i) ipilimumab, ii) a topoisomerase 1 inhibitor as a cytotoxic agent, and iii) a cleavable linker comprising a valine-citrulline dipeptide or a phenylalanine-lysine dipeptide.

A particular and preferred conjugate comprises i) tremelimumab, ii) a topoisomerase 1 inhibitor as a cytotoxic agent, and iii) a cleavable linker comprising a valine-citrulline dipeptide or a phenylalanine-lysine dipeptide.

A particular and preferred conjugate comprises i) ipilimumab, ii) SN38 as a cytotoxic agent, and iii) a cleavable linker comprising a valine-citrulline dipeptide or a phenylalanine-lysine dipeptide.

A particular and preferred conjugate comprises i) tremelimumab, ii) SN38 as a cytotoxic agent, and iii) a cleavable linker comprising a valine-citrulline dipeptide or a phenylalanine-lysine dipeptide.

A particular and preferred conjugate comprises i) ipilimumab, ii) exatecan as a cytotoxic agent, and iii) a cleavable linker comprising a valine-citrulline dipeptide or a phenylalanine-lysine dipeptide.

A particular and preferred conjugate comprises i) tremelimumab, ii) exatecan as a cytotoxic agent, and iii) a cleavable linker comprising a valine-citrulline dipeptide or a phenylalanine-lysine dipeptide.

A particular and preferred conjugate comprises i) ipilimumab, ii) a cytotoxic agent, preferably MMAF, and iii) a cleavable linker, preferably a linker comprising a valine-citrulline dipeptide such as the “Vc” dipeptide described herein below, or a phenylalanine-lysine dipeptide.

A particular and preferred conjugate comprises i) tremelimumab, ii) MMAF as a cytotoxic agent, and iii) a cleavable linker comprising a valine-citrulline dipeptide or a phenylalanine-lysine dipeptide.

A particular and preferred conjugate comprises i) ipilimumab, ii) MMAE as a cytotoxic agent, and iii) a cleavable linker comprising a valine-citrulline dipeptide or a phenylalanine-lysine dipeptide.

A particular and preferred conjugate comprises i) tremelimumab, ii) MMAE as a cytotoxic agent, and iii) a cleavable linker comprising a valine-citrullindipeptide or a phenylalanine-lysine dipeptide.

In another particular aspect, the cytotoxic agent is a DNA damaging agent such as an alkylating agent or a platinum complex.

Examples of alkylating agents include in particular Nitrogen mustards (e.g., bendamustine, chlorambucil, cyclophosphamide, ifosfamide, mechlorethamine, melphalan); Nitrosoureas (e.g., carmustine, lomustine, streptozocin); Alkyl sulfonates (e.g., busulfan); Triazines (e.g., dacarbazine, temozolomide); and Ethylenimines (e.g., altretamine, thiotepa).

Examples of platinum complexes include cisplatin, carboplatin and oxaliplatin.

In another particular aspect, the cytotoxic agent is a cytotoxic antibiotic such as for example bleomycin, a calicheamicin, daunorubicin, doxorubicin, dactinomycin, epirubicin, idarubicin, mitoxantrone, and mitomycin.

In another particular aspect, the cytotoxic agent is an antimetabolite such as for example 6-mercaptopurine, fludarabine, 5-fluorouracil, gemcitabine, cytarabine, pemetrexed, methotrexate).

In another particular aspect, the cytotoxic agent is a topoisomerase inhibitor such as for example Irinotecan (CPT-11) and Topotecan.

In another particular aspect, the cytotoxic agent is a RNA Polymerase Inhibitor such as for example Lurbinectedin and CX-5461.

In another particular aspect, the cytotoxic agent is an antimitotic agent, in particular a non-taxoid site microtubule-stabilizing agent such as for example Peloruside A (PLA) or laulimalide.

The payload may combine several distinct cytotoxic agents, for example any combination of the herein above described anticancer agents, in particular any combination of the herein above described cytotoxic agents.

In the context of the present invention, the drug-to-antibody ratio (DAR), i.e., the number of molecules of payload per antibody, may vary. The DAR is for example of two (DAR=2) or four (DAR=4).

### The Linker

The CTLA4 targeting molecule, for example the CTLA4 antibody, preferably the CTLA-4 monoclonal antibody, and the cytotoxic (anti-cancer) agent are linked together by a linker ensuring that less of the cytotoxic payload falls off before reaching a tumor cell, thereby improving safety and limiting dosages. The stability of the linker is critical in particular when the drug conjugate is administered to the subject in need thereof via the systemic route, typically by intravenous administration

Linkers are based on chemical motifs including disulfides, hydrazones or peptides (cleavable), or thioethers (non-cleavable). Cleavable and non-cleavable linkers were proved to be safe in preclinical and clinical trials [see for example brentuximab vedotin which includes an enzyme (cathepsin)-sensitive cleavable linker that delivers the antimicrotubule agent MMAE to human-specific CD30+ malignant cells, and trastuzumab emtansine which is a combination of a microtubule-formation inhibitor and of the trastuzumab antibody and which employs a stable, non-cleavable linker].

The availability of better and more stable linkers has changed the function of the chemical bond. The type of linker, cleavable or non-cleavable, lends specific properties to the (cytotoxic) payload. For example, a non-cleavable linker keeps the drug within the cell. As a result, the entire CTLA4 targeting molecule (for example the CTLA4 antibody including a CTLA4 monoclonal antibody), linker and payload enter the targeted CTLA4+cell, in particular the intratumoral Treg cell, where the CTLA4 targeting molecule is degraded. The resulting complex—CTLA4 targeting molecule, linker and cytotoxic agent—is considered to be the active drug. In contrast, cleavable linkers are detached by enzymes in the cell. The (cytotoxic) payload can then escape from the targeted cell and, in a process called “bystander killing”, attack neighboring cells.

A particular and preferred linker of the invention is a cleavable linker such as for example an acid-labile linker, an enzyme-cleavable linker, a lysosomal protease-sensitive linker, a disulfide linker or a β-glucuronide linker.

A preferred acid-labile linker is cleavable at the acidic pH existing within cellular endosomes.

A particular and preferred enzyme-cleavable linker comprises a valine-citrulline dipeptide (also identified in the experimental part as the “Vc” dipeptide) or a phenylalanine-lysine dipeptide.

A particular and preferred conjugate comprises i) ipilimumab, ii) a cytotoxic agent as herein described, and iii) a cleavable linker comprising a valine-citrulline dipeptide.

A particular and preferred conjugate comprises i) ipilimumab, ii) a cytotoxic agent as herein described, and iii) a cleavable linker comprising a phenylalanine-lysine dipeptide.

Another particular conjugate comprises i) tremelimumab, ii) a cytotoxic agent as herein described, and iii) a cleavable linker comprising a valine-citrulline dipeptide.

A further particular conjugate comprises i) tremelimumab, ii) a cytotoxic agent as herein described, and iii) a cleavable linker comprising a phenylalanine-lysine dipeptide.

### The Pharmaceutical Composition or Combination

A particular pharmaceutical composition of the invention comprises at least one drug conjugate according to the invention, for example at least two, three or four distinct drug conjugates, a first conjugate comprising a first payload (or cytotoxic agent), and a second or other (additional) conjugate comprising a distinct payload (or cytotoxic agent), and a pharmaceutically acceptable support, excipient, carrier, or diluent.

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable support, excipient, carrier and/or diluent. Thus, “Pharmaceutical composition” typically means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound, of the present invention, typically in a therapeutically effective amount, preferably at least one drug conjugate and at least one pharmaceutically acceptable carrier. A pharmaceutical composition of the present invention may additionally comprise one or more other compounds as active ingredients, such as one or more additional therapeutic, preferably anti-cancer, compounds of the present invention, or a prodrug compound or other known active substance, preferably active against cancer.

As used herein, an “effective dosage” or “effective amount” of a compound, for example of a drug conjugate, combination or composition is an amount sufficient to affect any one or more beneficial or desired outcomes, including biochemical, histological and/or behavioral symptoms, of the disease, typically cancer, and of its complications.

For a therapeutic use, a “therapeutically effective amount” refers to that amount of a compound, for example of a drug conjugate, combination or composition being administered which will relieve to some extent one or more of the symptoms of the disorder being treated. In reference to the treatment of cancer, a therapeutically effective amount refers to that amount which has the effect of reducing the size of the tumor, inhibiting (that is, slowing to some extent, preferably stopping) tumor metastasis, inhibiting to some extent (that is, slowing to some extent, preferably stopping) tumor growth or tumor invasiveness, relieving to some extent (or, preferably, eliminating) one or more signs or symptoms associated with the cancer, or associated to the treatment of cancer itself, decreasing the dose of other medications required to treat the disease or the secondary adverse (toxic) effects of the cancer treatment.

An effective dosage can be administered in one or more administrations. For the purposes of this invention, an effective dosage of drug, compound, (pharmaceutical) combination or (pharmaceutical) composition is an amount sufficient to accomplish prophylactic (in the context of the prevention of cancer relapse) or therapeutic treatment either directly or indirectly. As is understood in the clinical context, an effective dosage of a drug, compound, combination or composition may or may not be achieved in conjunction with another drug, compound, combination or composition.

As used herein, a “pharmaceutically acceptable support, excipient, carrier, or diluent” refers to a support, excipient, carrier, or diluent that does not cause significant irritation to an organism and does not abrogate the biological activity and properties of the active compound(s) or therapeutic agent(s). The pharmaceutical acceptable support, excipient, carrier, or diluent may comprise any conventional pharmaceutical support, excipient, carrier, or diluent. The choice of support, excipient, carrier or diluent will to a large extent depend on factors such as the particular mode of administration, the effect of the support, excipient, carrier or diluent on solubility and stability, and the nature of the dosage form. Suitable pharmaceutical carriers include inert diluents or fillers, water, and various organic solvents (such as hydrates and solvates). The pharmaceutical compositions may, if desired, contain additional ingredients such as flavorings, binders, and the like.

Another particular pharmaceutical composition of the invention comprises a drug conjugate, distinct therapeutic agent(s), and a pharmaceutically acceptable support, excipient, carrier, or diluent.

### Distinct Therapeutic Agents

As taught herein above, in a preferred aspect, the combination, composition, or herein below described kit, comprises, in addition to the CTLA4 targeting drug conjugate, at least one (i.e., one or more additional) distinct therapeutic agent, preferably anti-cancer agent(s).

The distinct therapeutic agent(s) is (are) typically anti-cancer agent(s). The anti-cancer agent(s) may be selected for example from an immune checkpoint targeting agent, an anti-angiogenic agent, a cytotoxic agent, an hormonal agent, an aromatase inhibitor, a glucocorticoid, and any combination thereof, wherein the amounts are preferably together effective in treating the cancer.

The immune checkpoint targeting agent can be an anti-PD1 agent [i.e., an agent targeting the Programmed Cell Death-1 receptor (PD1)] such as for example nivolumab, pembrolizumab, cemiplimab, retifanlimab, toripalimab or dostarlimab; an anti-PDL1 (Programmed Cell Death-1 receptor ligand) agent such as for example atezolizumab, durvalumab or avelumab; an anti-LAG3 (Lymphocyte-activation gene 3) agent such as relatlimab or eftilagimod; or an anti-TIGIT (T cell immunoreceptor with Ig and ITIM domains) agent such as tiragolumab or domvanalimab.

The anti-angiogenic agent may be a tyrosine kinase inhibitor such as for example sorafenib, sunitinib, vendetanib, lenvatinib, axitinib or cabozantinib.

The anti-angiogenic agent may be a monoclonal antibody directed against the vascular endothelial growth factor (VEGF) pathway (ligands or receptors), such as for example bevacizumab or aflibercept.

The cytotoxic agent may be an anti-cancer cytotoxic chemotherapeutic agent, for example an alkylating agent such as Nitrogen mustards (e.g., bendamustine, chlorambucil, cyclophosphamide, ifosfamide, mechlorethamine and melphalan); Nitrosoureas (e.g., carmustine, lomustine, streptozocin); Alkyl sulfonates (e.g., busulfan); Triazines (e. g, dacarbazine, temozolomide); Ethylenimines (e.g., altretamine, thiotepa); an antimetabolite such as 6-mercaptopurine, fludarabine, 5-fluorouracil, gemcitabine, cytarabine, pemetrexed, methotrexate; a topoisomerase inhibitor such as Irinotecan (CPT-11) or Topotecan; an antibiotic such as bleomycin, daunorubicin, doxorubicin, dactinomycin, epirubicin, idarubicin, mitoxantrone and mitomycin; or an antimitotic agent, in particular a non-taxoid site microtubule-stabilizing agent such as peloruside A (PLA) or laulimalide.

The hormonal agent is an agent inhibiting the estrogen or progesterone pathway such as tamoxifen.

The anti-cancer agent may be an aromatase inhibitor such as anastrozole (Arimidex) or exemestane (aromasin).

The anti-cancer agent may be a glucocorticoid pathway targeting agent such as prednisone, prednisolone, triamcinolone, methylprednisolone or dexamethasone.

In some embodiments, the additional anti-cancer agent is selected from the group consisting of an oncolytic virus (e.g., T-VEC), an oncolytic peptide (e.g., LTX315) and a protein kinase C agonist (e.g., tigilanol tiglate).

In a preferred embodiment, the at least one distinct therapeutic agent, preferably anti-cancer agent, is selected from an immune checkpoint targeting agent, a cytotoxic agent, in particular an anti-cancer cytotoxic chemotherapeutic agent, an anti-angiogenic agent, an hormonal agent and any mixture or combination thereof.

### Route of Administration and Formulations

The (pharmaceutical) combinations or (pharmaceutical) compositions of the invention include combinations and compositions suitable for oral, rectal, topical, parenteral (including subcutaneous, intramuscular and intravenous), ocular (ophthalmic), pulmonary (nasal or buccal inhalation), or nasal administration, although the most suitable route in any given case will depend on the nature and severity of the conditions being treated and on the nature of the active ingredient(s). Any suitable route of administration may be employed for providing a mammal, especially a human, with an effective dose of an active compound of the present invention. In the context of the present invention, active compounds are preferably administered intravenously and/or intra-tumorally. A preferred route of administration for the herein described drug conjugate is the intra-tumoral route.

The active ingredients may be conveniently presented in unit dosage form and prepared by any of the methods well-known in the art of pharmacy. Dosage forms include for example tablets, dispersions, suspensions, solutions, capsules, creams, ointments, aerosols, and the like.

The (pharmaceutical) combination or pharmaceutical composition may be in a form for example suitable for oral administration as a tablet, capsule, pill, powder, sustained release formulation, solution or suspension, suitable for parenteral injection as a sterile solution, suspension or emulsion, suitable for topical administration as an ointment or cream, or suitable for intratumoral administration as a sterile solution, suspension, emulsion, or slow release chemical formulation allowing for an augmented stay and a slow release of the drug conjugate, in particular of the CTLA4 ADC, in the tumor micro-environment.

The combination or pharmaceutical composition may be in unit dosage forms suitable for single administration of precise amounts.

Combination or pharmaceutical compositions suitable for the delivery of the therapeutic agents of the combination therapies of the present invention, and methods for their preparation will be readily apparent to those skilled in the art. Such compositions and methods for their preparation may be found, for example, in ‘Remington's Pharmaceutical Sciences’, 19th Edition (Mack Publishing Company, 1995), the disclosure of which is incorporated herein by reference in its entirety.

Suitable modified release formulations are described in U.S. Pat. No. 6,106,864. Details of other suitable release technologies such as high energy dispersions and osmotic and coated particles may be found in Verma et al., Pharmaceutical Technology On-line, 25(2), 1-14 (2001). The disclosures of these references are incorporated herein by reference in their entireties.

In practical use, the compounds of the present invention can be combined as the active ingredient in intimate admixture with a pharmaceutical support, excipient, carrier and/or diluent according to conventional pharmaceutical compounding techniques. As herein above explained, the support, excipient or carrier may take a wide variety of forms depending on the form of preparation desired for administration, e.g., oral or parenteral (including intravenous or intra-tumoral).

### Posology

When treating or preventing a cancer mentioned herein with a drug conjugate, generally satisfactory results are obtained when said drug conjugate is administered at a dosage of from about 1,5mg/kg milligrams (mg) to about 5,5 mg/kg, possibly given as a single dose delivered IV every 3 weeks (Q3W).

The daily dosage of the anti-CTLA4 or CTLA4 targeting drug conjugate administered to the subject is for example of about 0,01 mg/kg to about 10 mg/kg, preferably of about 0,1 mg/kg to about 1 or 3 mg/kg, and even more preferably of about 0,1 mg/kg to about 1 mg/kg.

An effective dosage of a CTLA4 targeting drug conjugate of the invention is typically in the range of from about 0,01 mg to about 5 or to about 10 mg per kg body weight, preferably from about 0,01 to about 1 mg/kg body weight, in single or divided doses. This dosage may be administered to the subject in need thereof per day or per week or every 2 weeks or every 3 weeks. This dosage regimen may be adjusted by the oncologist to provide the optimal therapeutic response to the patient.

In some instances, drug conjugate's dosage levels at the lower limit of the aforesaid range may be more than adequate, while in other cases still larger doses may be employed without causing any harmful side effect, provided that such larger doses are first divided into several small doses for administration throughout the day. The dosage may be administered as a single dose, or optionally may be subdivided into smaller doses, suitable for a twice-daily, three times-daily or four times-daily administration.

The therapeutically effective amount of a compound depends on a number of factors, including, for example, the age and weight of the (mammal) subject, the precise condition that requires treatment, and its severity, the nature of the formulation and the method of administration, and is ultimately determined by the treating doctor or vet. However, an effective amount of a CTLA4 targeting drug conjugate according to the invention is generally in the range from 0,01 to 5 or to 10 mg/kg of body weight of the recipient (mammal subject) per day and particularly typically in the range from 0,01 to 1 mg/kg of body weight per day. Thus, the actual amount per day for an adult human weighing 70 kg is usually between 0,7 and 70 mg, where this amount can be administered as a single dose per day or usually in a series of part-doses (such as, for example, two, three, four, five or six) per day, so that the total daily dose is the same.

The effective dosage of any active ingredient herein described may vary depending on the particular compound employed, the mode of administration, the condition of treatment and the severity of the condition being treated. Such dosage may be ascertained readily by a person skilled in the art, typically by an oncologist.

In a particular aspect, the drug conjugate comprises i) ipilimumab or tremelimumab, ii) a cytotoxic agent, such as for example a microtubulin inhibitor, a maytansinoid, a DNA binding agent or a toposisomerase inhibitor, in particular for example MMAE, MMAF, DM1, DM4, calicheamicin, SN38 or exatecan, preferably anyone of DM1, DM4, MMAE or MMAF, even more preferably MMAF, and iii) a cleavable linker, preferably a linker comprising a valine-citrulline dipeptide or a phenylalanine-lysine dipeptide, and said drug conjugate is present in the combination or composition at a dose of about 0,01 mg to about 10 mg, preferably about 0,1 mg to about 1 mg, for example 0,2 mg, 0,3 mg, 0,4 mg, 0,5 mg, 0,6 mg, 0,7 mg, 0,8 mg or 0,9 mg, and more preferably about 0,3 mg.

An effective dosage of a ipilimumab is typically in the range of from about 1 to about 10 mg per kg body weight, preferably about 3 to about 10 mg/kg, for example 4 mg/kg, 5 mg/kg, 6 mg/kg, 7 mg/kg, 8 mg/kg or 9 mg/kg, in single or divided doses, typically per day.

In a particular aspect, the drug conjugate comprises i) ipilimumab or tremelimumab, ii) a cytotoxic agent, such as for example a microtubulin inhibitor, a maytansinoid, a DNA binding agent or a toposisomerase inhibitor, in particular for example MMAE, MMAF, DM1, DM4, calicheamicin, SN38 or exatecan, preferably anyone of DM1, DM4, MMAE or MMAF, and iii) a cleavable linker, preferably a linker comprising a valine-citrulline dipeptide or a phenylalanine-lysine dipeptide, and said drug conjugate is present in the combination or composition at a dose of about 0,01 mg to about 10 mg, preferably about 0,1 mg to about 1 mg, for example 0,2 mg, 0,3 mg, 0,4 mg, 0,5 mg, 0,6 mg, 0,7 mg, 0,8 mg or 0,9 mg, and more preferably about 0,3 mg.

An effective dosage of a ipilimumab is typically in the range of from about 1 to about 10 mg per kg body weight, preferably about 3 to about 10 mg/kg, for example 4 mg/kg, 5 mg/kg, 6 mg/kg, 7 mg/kg, 8 mg/kg or 9 mg/kg, in single or divided doses, typically per day.

### The Kits

The kits described herein may be particularly suitable for administering different dosage forms, for example, oral and parenteral (typically intravenous or intra-tumoral, preferably intra-tumoral), for administering the separate active (in particular therapeutic) agents of the combinations or compositions at different dosage intervals, or for titrating the active (in particular therapeutic) agents of the combination or compositions against one another. To assist compliance, the kit typically includes instructions for administration. The kit may further comprise other materials that may be useful in administering the medicaments, such as diluents, filters, IV bags and lines, needles and syringes, and the like.

The kit of the invention comprises at least two drug conjugates as herein described, or at least one drug conjugate as herein described and distinct therapeutic agent(s) as herein described, for example selected from an immune checkpoint targeting agent, an anti-angiogenic agent, a cytotoxic agent, an hormonal agent and any combination thereof.

A particular kit comprises a) at least a drug conjugate comprising a CTLA4 targeting molecule as herein described, in particular a drug conjugate, in particular an anti-CTLA4 ADC, comprising i) a CTLA4 targeting molecule, preferably an anti-CTLA-4 monoclonal antibody, ii) at least one cytotoxic agent, and iii) a linker connecting the CTLA4 targeting molecule and the cytotoxic agent, and b) distinct therapeutic agent(s) as herein described, for example selected from an immune checkpoint targeting agent, an anti-angiogenic agent, a cytotoxic agent, an hormonal agent and a combination thereof.

Another particular kit comprises a pickering emulsion of a CTLA4 targeting molecule, in particular an anti-CTLA4 ADC, formulated in poly-lactic-co-glycolic acid (PLGA) nanoparticles, with or without radiopaque ethiodized oil, in order to allow a prolonged stay and slow release of the drug conjugate in the injected tumors.

### Therapeutic Uses of the Drug Conjugate, Combinations, Compositions and Kits

Inventors also describe a combination or composition as described herein for use as a medicament. The combination or composition of the invention comprises at least a CTLA4 targeting molecule as herein described, preferably a drug conjugate comprising i) a CTLA4 targeting molecule, preferably an anti-CTLA-4 monoclonal antibody, ii) at least one cytotoxic agent such as for example a microtubulin inhibitor, a maytansinoid, a DNA binding agent or a toposisomerase inhibitor, in particular for example MMAE, MMAF, DM1, DM4, calicheamicin, SN38 or exatecan, and iii) a linker, preferably a cleavable linker as herein described such as a valine-citrulline dipeptide or a phenylalanine-lysine dipeptide, connecting the CTLA4 targeting molecule and the cytotoxic agent. The combination or composition of the invention typically comprises additional (distinct) therapeutic agent(s). The several distinct (active) agents may be used sequentially, simultaneously or concurrently in the subject in need thereof.

As used herein, the terms “combination” or “combination therapy” refer to the administration of each therapeutic agent(s) of the combination therapy of the invention, either alone or in the form of a pharmaceutical composition or medicament, either sequentially, concurrently, or simultaneously.

As used herein, the term “sequential” or “sequentially” refers to the administration of each therapeutic agent(s) of the combination therapy of the invention, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

As used herein, the term “concurrently” refers to the administration of each therapeutic agent in the combination therapy of the invention, either alone or in separate medicaments, the second therapeutic agent being administered immediately after the first therapeutic agent, and the therapeutic agents being administered in any order. In a preferred embodiment, the therapeutic agents are administered concurrently.

As used herein, the term “simultaneous” refers to the administration of each therapeutic agent of the combination therapy of the invention in the same medicament. As will be understood by those skilled in the art, the combination therapy may be usefully administered to a subject during different stages of the treatment.

The drug conjugate, composition or kit of the invention is for use for treating (or used in the context of the treatment of) cancer, or is for use for preventing (or used in the context of the prevention of) a cancer relapse in a subject in need thereof.

In the context of the present invention, the term “patient” and “subject” are synonyms and refer to any single subject for which therapy is desired or that is participating in a clinical trial, epidemiological study or used as a control, including humans and mammalian veterinary patients.

The “subject” or “patient” is typically a mammal. The subject can be a human or a non-human mammal such as a rodent, for example a mouse or a rat; a rabbit; a primate such as a monkey; a dog, a cat, a bovid, an equine, for example a horse; or transgenic species thereof.

In a particular and preferred aspect, the mammal is a human being, whatever its age or sex. In a particular aspect, the subject is an adult human subject. In another particular aspect, the subject is a human child between the ages of birth and 18 years old.

In a particular aspect of each of the herein described methods, combinations and uses, the drug conjugate of the invention is administered to a subject, in particular to a subject diagnosed as suffering of cancer, who is previously untreated (for cancer), i.e. who is cancer treatment naïve. In another particular aspect of each of the herein described methods, combinations and uses, the drug conjugate of the invention is administered to a subject suffering of cancer who has been exposed to at least one prior therapy with an anti-cancer agent, in particular to a prior therapy comprising an anti-CTLA-4 monoclonal antibody such as any monoclonal antibody herein described, in particular ipilimumab or tremelimumab [used alone or in combination with an additional conventional anti-cancer agent such as an anti-PD1 (for example nivolumab)], or whose tumor(s) has (have) been surgically resected, i.e., who is a treatment experienced-subject. In a particular and preferred aspect of each of the herein described methods, combinations and uses, the drug conjugate of the invention is administered to a subject, in particular to a subject diagnosed as suffering of cancer, preferably in a neoadjuvant setting (i.e. before any surgery). Thus, in a preferred aspect, the malignant tumor, in particular primary malignant tumor and/or draining lymph nodes, has (have) not been surgically resected.

In another particular aspect, the subject has been exposed for example to part of a complete conventional treatment protocol, for example to at least one cycle of the total planned treatment protocol. In again another aspect, the subject has been exposed to a complete conventional protocol.

Patients are preferably treated with the combination or composition of the invention when disease progression and/or an unacceptable therapy induced-toxicity are observed or are expected.

In particular aspects of each of the herein described drug conjugates, compositions, kits, combinations, methods and uses, the subject's cancer is a cancer as herein above defined, in particular a solid cancer selected from melanoma, renal cell carcinoma (RCC), colorectal cancer, hepatocellular carcinoma, non-small cell lung cancer (NSCLC), mesothelioma (MM) in particular pleural mesothelioma, or a hematological malignancy selected from a lymphoma and a leukemia.

In the context of the herein described drug conjugates, compositions, kits, combinations, methods and uses, the subject's cancer is either an early stage cancer such as a localized primary tumor, or an advanced cancer or cancer with poor prognosis such as a locally advanced cancer, a (surgically) inoperable cancer, a metastatic cancer, a recurrent cancer, or a cancer resistant to a conventional therapeutic treatment.

In a particular aspect of the invention, the drug conjugate, composition, combination or kit is for use (used) in combination with a radiation therapy (radiotherapy), i.e., is combined with the exposition of the subject's tumor(s) to radiations.

The radiation therapy can be for example external-beam radiation therapy, brachytherapy, systemic radiation therapy, notably radio-ligand based radiation therapy, or proton therapy.

The invention beneficiates in particular to the subjects suffering of a cancer whose tumor(s) or cancer tumor microenvironment (TME) comprise(s) CTLA4+ cells (i.e., CTLA4 expressing cells), in particular CTLA4+ tumor cells and/or CTLA4+ immune cells, preferably CTLA4+ Tregs. Such tumors are herein considered as being in an “immuno-editing stage”, i.e., as tumors particularly sensitive to (/able to beneficiate from) an anti-CTLA4 drug conjugate of the invention.

The invention also beneficiates in particular to the subjects who, in addition of suffering of a cancer whose tumor(s) or cancer tumor microenvironment (TME) comprise(s) CTLA4+ cells (in particular CTLA4+ tumor cells and/or CTLA4+ immune cells, preferably CTLA4+ Tregs), do not or will not exhibit a “Durable Clinical Benefit” upon (naked) anti-CTLA4 and/or anti-PD(L)1 therapy (“DCB” meaning being in complete response or partial response or stable disease according to RECIST 1.1 criteria at 6 months post treatment initiation). These patients are also herein identified as “no Durable Clinical Benefit” (or “no DCB”) patients.

In other words, these subjects or patients are either patients naïve to anti-cancer treatment or patients who have been exposed to anti-cancer treatment but exhibit resistance (primary or secondary resistance, preferably primary resistance) to said treatment, in particular to (naked) anti-CTLA4 and/or anti-PD(L)1.

In a particular aspect, the subject naïve to (anti-cancer) treatment is a subject predicted to be resistant to (naked) anti-CTLA4 and/or anti-PD(L)1 agent(s).

A particular predicted “no DCB” subject or patient is a subject whose tumor comprises no, low level of, or dysfunctional myeloid cells, in particular macrophages, said myeloid cells expressing no or low level of the Fcγ receptor I (“CD64”), Fcγ receptor IIIa (“CD16a”) and/or Fcγ receptor IIIb (“CD16b”).

In the context of the present invention, a low level of the Fcγ receptor I (“CD64”), Fcγ receptor IIIa (“CD16a”) and/or Fcγ receptor IIIb (“CD16b”) expressed by myeloid cells is a level insufficient to allow an ADCC or ADCP activity.

A dysfunctional myeloid cell is herein defined as a cell which is not able to perform an antibody derived cell cytotoxicity (ADCC) or antibody derived cell phagocytosis (ADCP). In vitro ADCC/ADCP tests may be performed by the skilled person in the art according to know methods (cf. Yamashita, M., Kitano, S., Aikawa, H. et al. A novel method for evaluating antibody-dependent cell-mediated cytotoxicity by flowcytometry using cryopreserved human peripheral blood mononuclear cells. Sci Rep 6, 19772 (2016)).

Another particular predicted “no DCB” subject or patient is a subject whose tumor comprises a proportion of myeloid cells, in particular macrophages, positive for Fcγ receptor I (“CD64”), Fcγ receptor IIIa (“CD16a”) and/or Fcγ receptor IIIb (“CD16b”) inferior to the proportion detected in a cancerous patient, or population of cancerous patients, suffering from the same cancer identified, or predicted, as exhibiting a durable clinical benefit for an anti-cancer treatment, preferably an anti-cancer treatment involving in particular an anti-CTLA-4 monoclonal antibody such as for example ipilimumab or tremelimumab [used alone or in combination with an additional conventional anti-cancer therapeutic agent such as an anti-PD1 (for example nivolumab)].

Thus, the “no DCB” subjects are preferred subjects in the context of the present invention. Indeed, these no DCB patients are particularly likely to benefit from the ADC of the present invention since said ADC is capable of generating a clinical benefit in said patients, or in other words is capable of turning a “no DCB” subject or patient into “DCB” subject or patient. In these DCB patients, a stabilization, or a partial or total regression, of the disease will be advantageously observed thanks to the present invention.

Particular CTLA4 expressing cells sensitive to the herein described anti-CTLA4 drug conjugate are regulatory T-cells, preferably CD4+FOXP3+CTLA4+ T cells and/or CD4+CD25+CD39+ T cells. These Treg cells are clonally expanding Treg cells specific for the tumor antigen(s). Preferred subjects are subjects suffering from a cancer whose tumor(s) and/or TME comprise(s) such CTLA4 expressing cells. Those subjects are the more likely to benefit of the administration of a drug conjugate or composition comprising such a drug conjugate of the present invention, in particular a drug conjugate comprising i) a Cytotoxic-T-Lymphocyte-Antigen 4 protein (CTLA4) targeting molecule, preferably an anti-CTLA-4 monoclonal antibody, ii) at least one cytotoxic agent, and iii) a linker connecting the CTLA4 targeting molecule and the cytotoxic agent.

The therapeutic targeting of such a sub-population of tumor or immune cells, in particular the therapeutic targeting of tumour antigen-specific Tregs allow the development of advantageous specific anti-tumour immune response(s) to cancer while preserving (i.e., being less toxic) to the healthy tissue, typically to the healthy tissue surrounding the tumor(s), in particular if the anti-CTLA4 drug conjugate or composition comprising said drug conjugate is intra-tumorally administered.

Thus, in a particular aspect of the present invention, the cancerous subject to be treated is a subject whose tumor or Tumor Micro Environment (TME) comprises CTLA4+ cells, preferably CTLA4+ tumor cells and/or CTLA4+ immune cells, notably CTLA4+ Tregs.

In another aspect, the subject is a subject having resistance to (naked) anti-CTLA4 and/or anti-PD(L)1 agent(s).

In again another particular aspect of the present invention, the cancerous subject to be treated is a subject whose tumor and/or TME display(s) no, low level of, or dysfunctional myeloid cells, said myeloid cells expressing no or low levels of the Fcγ receptor I (“CD64”), Fcγ receptor IIIa (“CD16a”) and/or Fcγ receptor IIIb (“CD16b”), in particular a subject whose tumor or TME does not comprise myeloid cells, in particular macrophage, expressing no or low levels of the Fcγ receptor I (“CD64”), Fcγ receptor IIIa (“CD16a”) and/or Fcγ receptor IIIb (“CD16b”).

In another aspect, the subject to be treated is a subject who comprises a proportion of myeloid cells, in particular macrophages, positive for the Fcγ receptor I (“CD64”), Fcγ receptor IIIa (“CD16a”) and/or Fcγ receptor IIIb (“CD16b”) inferior to the proportion detected in a cancerous patient, or population of cancerous patients, suffering from the same cancer identified, or predicted, as exhibiting a durable clinical benefit (DCB) for an anti-cancer treatment, preferably an anti-cancer treatment, involving in particular an anti-CTLA-4 monoclonal antibody such as for example ipilimumab or tremelimumab [used alone or in combination with an additional conventional anti-cancer therapeutic agent such as an anti-PD1 (for example nivolumab)].

Herein described is also a method to select/identify the subjects, typically patients, most likely to be sensitive, or more responsive, to the herein described cancer treatments, in particular to be rendered sensitive again, or more responsive, to a treatment of cancer, for example after having shown resistance to anti-cancer treatment, or having been identified or predicted as “no DCB” subjects. Said method preferably comprises a step i) of determining the presence or absence of CTLA4+ cells in the tumor or in the TME (said cells being also identified as “intratumoral CTLA4+ cells”), and also possibly the presence or absence of myeloid cells in the tumor or in the TME (said cells being also identified as “intratumoral myeloid cells”), notably macrophages, and/or the expression level by said myeloid cells of the Fcγ receptor I (“CD64”), Fcγ receptor IIIa (“CD16a”) and/or Fcγ receptor IIIb (“CD16b”), and a step ii) of identifying said subject as a subject who will particularly beneficiate of a treatment as herein described comprising the administration of a drug conjugate of the invention.

The detection of CTLA4+ cells may be easily performed in vitro, ex vivo or in vivo, by the person of ordinary skill in the art with any commercially available anti-CTLA4 staining antibody, such as Biolegend ref 369612 (BD Biosciences ref 557301).

As well, the detection and quantification of myeloid cells, notably macrophages expressing the Fcγ receptor I (“CD64”), Fcγ receptor IIIa (“CD16a”) and/or Fcγ receptor IIIb (“CD16b”) may be easily performed on fixed or frozen baseline tumor biopsies from cancerous patients in vitro, by the person of ordinary skill in the art with a transcriptomic assessment method such as RNAseq or qRT-PCR used to detect or quantify expression levels of anyone of the following genes: CD68, CD163, CSF1R, CD16a, CD16b and CD64, or any combination thereof, in particular CD64, CD16a and/or CD16b.

The drug conjugate or composition of the invention may be administered to the subject as a neoadjuvant therapeutic agent, preferably before any partial or total tumor surgical resection or focal destruction by any cytoreductive strategy, in monotherapy or in combination with distinct anti-cancer therapeutic agent(s), and preferably by local delivery including intra-tumoral (IT) route, intra-vascular (IV) route or topical route, via a single administration or via repeated administrations.

The drug conjugate of the invention can be advantageously administered intratumorally to cancer patients, in particular to patients having localized solid cancers, preferably before any cancer treatment, in particular prior surgery.

In a preferred aspect, the intra-tumorally administered anti-CTLA4 drug conjugate, in particular anti-CTLA4 ADC, herein described, maximizes the bioavailability and therefore the local efficacy of the treatment of cancer, in particular of the anti-cancer payload, by inducing the depletion of CTLA4+ immune cells in the tumor and/or tumor microenvironment while avoiding any anti-cancer treatment systemic toxicity (by considerably lowering the systemic exposure). As indicated herein above, the drug conjugate of the invention can be also advantageously administered to cancer patients having metastatic malignant tumors.

A drug conjugate for use as a medicament, preferably for treating cancer, is advantageously herein above described by inventors. Also herein described are the corresponding therapeutic uses, in particular the corresponding methods for treating cancer or preventing cancer relapse in a subject in need thereof. These methods comprise a step of administering the drug conjugate or composition of the invention comprising said drug conjugate to the subject in need thereof, alone or in combination with one or several distinct therapeutic compounds such as those herein described. A preferred drug conjugate comprises i) a CTLA4 targeting molecule, in particular an anti-CTLA4 monoclonal antibody such as ipilimumab or tremelimumab, ii) at least one cytotoxic agent such as for example a microtubulin inhibitor, a maytansinoid, a DNA binding agent or a toposisomerase inhibitor, in particular for example MMAE, MMAF, DM1, DM4, calicheamicin, SN38 or exatecan, and iii) a linker, preferably a cleavable linker as herein described such as a valine-citrulline dipeptide or a phenylalanine-lysine dipeptide, connecting the CTLA4 targeting molecule and the cytotoxic agent.

### FIGURES

FIG. 1. Impact of the anti-CTLA4 ipilimumab (IPI) addition to the anti-PD1 nivolumab (NIVO) on the complete response rate in metastatic Melanoma is cancer stage dependent. Pooled results from CheckMate 066, 067, 069 trials from Robert, C. et al. 1082MO 5 year characterization of complete responses in patients with advanced melanoma who received nivolumab plus ipilimumab (Nivo+Ipi) or Nivo alone. Ann. Oncol. 31, S734-S735 (2020).

FIG. 2. CTLA4 membrane expression on intratumoral T-cells.

(a) Proportions and Mean Fluorescent Intensity (“MFI”) of membrane CTLA4 expression on T cells populations (b) CTLA4+ Treg proportion among cancer types (MM: Metastatic Melanoma; NSCLC: Non-Small Cell Lung Cancer; RCC: Renal Cell Cancer; HNSCC: Head and Neck Squamous Cell Carcinoma; EOC: Epithelial Ovarian Cancer) (c) Proportions of CTLA4+ cells among intra-tumoral FoxP3+ CD4+ T-cells and (d) Proportions of CTLA4+ cells among intra-tumoral CD25+ FoxP3+ CD4+ T-cells upon co-incubation of freshly resected and dissociated human tumors with ipilimumab or its isotype. (e) Illustration of the proportion of CTLA4+ FOXP3+ CD4+ T-cells and the CTLA4 MFI from one tumor. All measures were obtained by non-competitive staining of CTLA4 by flow cytometry.

FIG. 3. Ipilimumab cellular distribution at 37 °C in comparison to 4 °C, confocal microscopy.

At 37 °C, Ipilimumab is internalized and localized in sub-membrane intracellular vesicles within 1 hour on CTLA4 expressing cells. Diffused Ipilimumab intracellular staining is observed upon 24hours.

FIG. 4. Intracytoplasmic FoxP3 and Membrane CTLA4 labeling by flow cytometry among CD4+ CD25+ CD39+ populations (double positive, single positive, or double negative) of freshly dissociated melanoma tumors.

FIG. 5. CD4+ CD25+ CD39+ Intratumoral Tregs Significantly Diminish in Proportion upon intratumoral ipilimumab treatment.

Left panel: Flow cytometry of immune cells performed on fresh biopsy samples showing that at baseline (prior to any treatment), patients having a high proportion of CD4+CD39^{high}CD25^{high} T cells are significantly more abundant in melanoma patients who will develop durable clinical benefit from the treatment (DCB: disease control lasting for more than 6 months). Middle and Right panels: this intratumoral CD4+CD25+CD39+ T cells population is significantly diminished upon ipilimumab administration when ipilimumab is administered intratumorally into melanoma patients (IT arm) and to a lower extent when ipilimumab is administered in melanoma patients intravenously (IV arm).

FIG. 6. Single Cell Sequencing of Enriched CD45+ Cells From Five Freshly Resected Tumors.

(a) clonality among the 4 main subsets of T cells; (b) specific T cells clustering; (c) subset of specific T cells.

FIG. 7. Intratumoral ipilimumab generates less toxicity in patients than intravenous ipilimumab while maintaining the same anti-tumor efficacy.

Proportions of treatment related adverse events according to treatment arm (a) and best response according to recist1.1 (b).

FIG. 8: Generation of 3 Ipilimumab Antibody-drug Conjugates.

(a) chemical formula of the 3 payloads; (b) Principles of UV light binding of payloads to ipilimumab via the valine-citrulline linker; (c) SDS reducing gel confirming the molecular mass shift upon Ipilimumab binding to payloads. Lanes left to right are 1: DM1, 2: DM1+Ipilimumab, 3: DM1+Rituximab, 4: VeMMAE, 5: VcMMAE+Ipilimumab, 6: VcMMAE+Rituximab, 7: VcMMAF, 8: VcMMAF+Ipilimumab, 8 VcMMAF+Rituximab, 10: Ladder, 11: Ipilimumab, 12: Rituximab

FIG. 9: Phenotype of Raji hCTLA4 and Raji cell lines.

CTLA-4 expression at the membrane of Raji and RaijhCTLA-4 cells

FIG. 10: Cytotoxicity induced by in vitro ipilimumab ADC treatments on a cell line overexpressing hCTLA-4 (a, b, c) and on a control cell line which does not express CTLA4 (d).

Percent of dead cells at different time points and concentrations of ADC treatments (DM1_Ipilimumab, VcMMAE_Ipilimumab, VcMMAF_Ipilimumab) on Raji hCTLA4 cell line after (a) 48 hours of treatment (n=3), (b) 72 hours of treatment (n=4), (c) 96 hours of treatment (n=3), and (d) on a control cell line, Raji without CTLA4 expression, after 96 hours of treatment (n=3).

FIG. 11: Structural side scatter representation of RajihCTLA4 cells treated with increasing concentrations of Ipilimumab-VcMMAF, Rituximab-VcMMAF, Ipilimumab, Rituximab and VcMMAF.

(a) FSC/SSC of Raji hCTLA4; (b) proportions of dead cells among both populations; (c) CTLA4 expression of both populations; (d) FSC/SSC dot plots of RajihCTLA4 cells treated with Ipilimumab-VcMMAF, Rituximab-VcMMAF, Ipilimumab, Rituximab and VcMMAF during 72 hours.

FIG. 12: The tumor microenvironment of patients resistant to ipilimumab are devoid of adequate macrophages. The tumor microenvironment of patients resistant to ipilimumab are devoid of adequate macrophages (CD68 and/or CD163 and/or CSF1R) and FcgRI (CD64) and/or FcgRIIIa (CD16a) and/or FcgRIIIb (CD16b). Bar charts representing comparison at baseline of transcripts per million (TPM) of CD68, CD163, CSF1R, CD16a and CD16b and CD64 between patient with clinical benefits (DCB) versus non clinical benefits (noDCB). The results presented here are from patients with metastatic melanoma who were enrolled in a phase 1 study and received an initial combination of ipilimumab and nivolumab. DCB=clinical benefit at 6 months.

Throughout this application, various references describe the state of the art to which this invention pertains. The disclosures of these references are hereby incorporated by reference into the present disclosure.

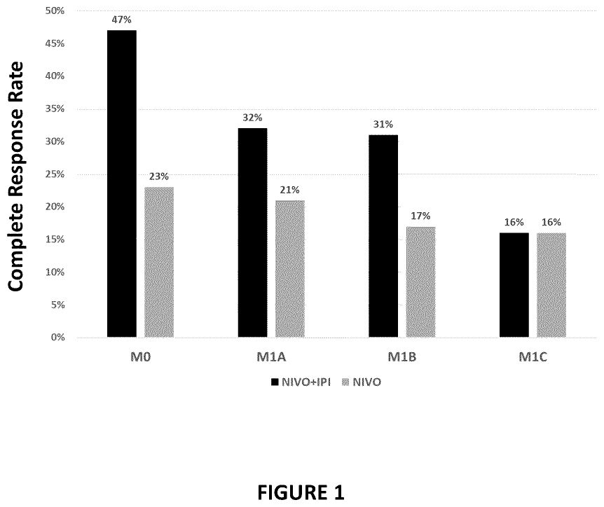

Other characteristics and advantages of the invention are given in the following experimental section (with reference to FIGS. 1 to 12), which should be regarded as illustrative and not limiting the scope of the present application.

### Experimental Part

### Development OF AN Anti-CTLA4 Drug Conjugate for Intratumoral Administraton

### Material and Methods

### Antibody Drug Conjugate

Ipilimumab and Rituximab were labeled to three different cytotoxic drug payloads (DM1, VeMMAE, VcMMAF) following the manufacturer (oYoLink®'s Alpha thera) protocols. Each different payload is linked via the lysosomally cleavable dipeptide, valine-citrulline (“vc” or “Vc”).

### Cell Cultures

Raji hCTLA-4 cell line has been grown with Iscove's Modified Dulbecco's Medium (Sigma Life Sciences, ref: 13390-500mL) supplemented with 10% decomplemented SVF Hyclone (Research Grade, ref: SV 30160.03), 1% L-Glutamine (Gibco, ref: 25030-024), 25nM Hepes (Gibco, ref: 15630-056), 1% Penicillin/Streptomycin (Gibco, ref: 15140-122), 10 ug/ml of Blasticidin (Invivogen, ref: ant-bl-1). Raji cell line has been grown with IMDM (Sigma Life Sciences, ref: 13390-500 mL) supplemented with 10% decomplemented SVF Hyclone (Research Grade, ref: SV30160.03), 1% L-Glutamine (Gibco, ref: 25030-024), 1% Penicillin/Streptomycin (Gibco, ref: 15140-122).

### Cytotoxicity Assays

The proportion of dead cells was estimated by flow cytometry using Zombie Aqua (Biolegend) staining of dead cells and expressed as the percentage of dead cells in all events excluding cell debris. The cells are incubated at room temperature during 20 minutes and then washed with 2 ml of PBS.

In order to evaluate the most effective anti-CTLA4 drug conjugate concentrations, inventors compared the percentage of dead cells induced by concentrations between 0 and 1uM of Ipilimumab-Drug conjugates to the Rituximab-Drug conjugates, to Ipilimumab, to Rituximab and to the payloads alone at different time points: 48 hours, 72 hours and 96 hours.

They performed an additional control by treating a Raji cell line not expressing CTLA4 during 96 hours with the same experimental conditions (Ipilimumab drug conjugates, Rituximab drug conjugates, Rituximab, Ipilimumab, and payloads alone, at the same concentrations).

### Results

CD4+FOXP3+T cells called regulatory T cells or “Tregs” are a subgroup of lymphocytes with a key role in generating immune system tolerance (Lucca and Dominguez-Villar, 2020; Plitas and Rudensky, 2020). Their presence or function in excess or deficiency has been associated with autoimmune diseases or cancers respectively (Tay et al., 2023). CTLA4 is a co-inhibitory (“checkpoint”) receptor expressed at the membrane level by Tregs after engagement of their TCR receptor (Rowshanravan et al., 2018). Consensus on the mechanism of action of anti-CTLA4 molecules in mice points to a specific depletion of tumour Tregs (Marabelle et al., 2013; Selby et al., 2013; Simpson et al., 2013). However, in humans, the subject remains controversial between a CTLA4 antagonist role and the destruction of Tregs (Ferrara et al., 2019; Sharma et al., 2019). The impact of the level of disease progression (“cancer stage”) on the efficacy of anti-CTLA4 has also been shown recently by the rate of complete responses to anti-PD1+/-anti-CTLA4 in melanoma (FIG. 1).

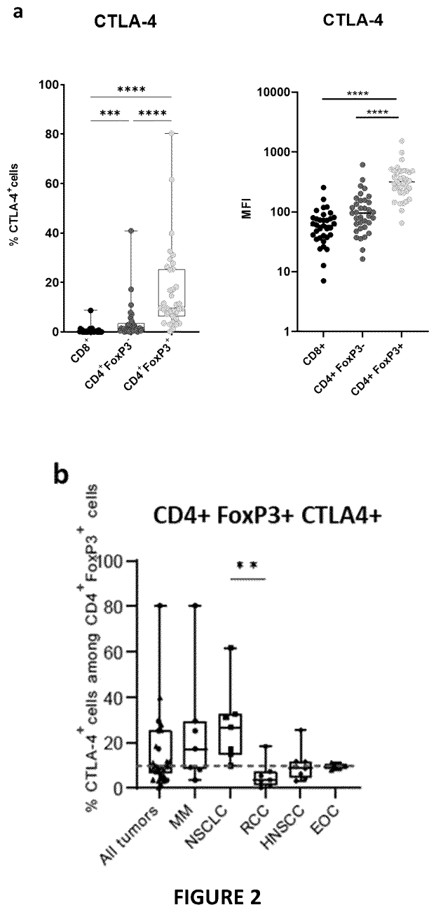

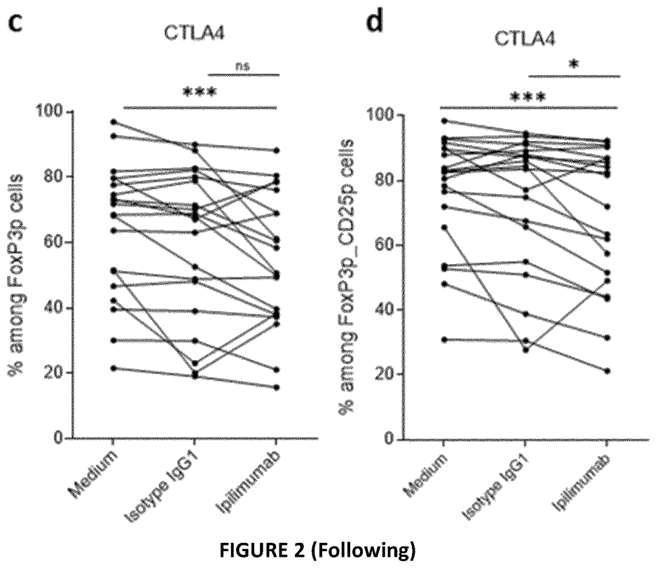

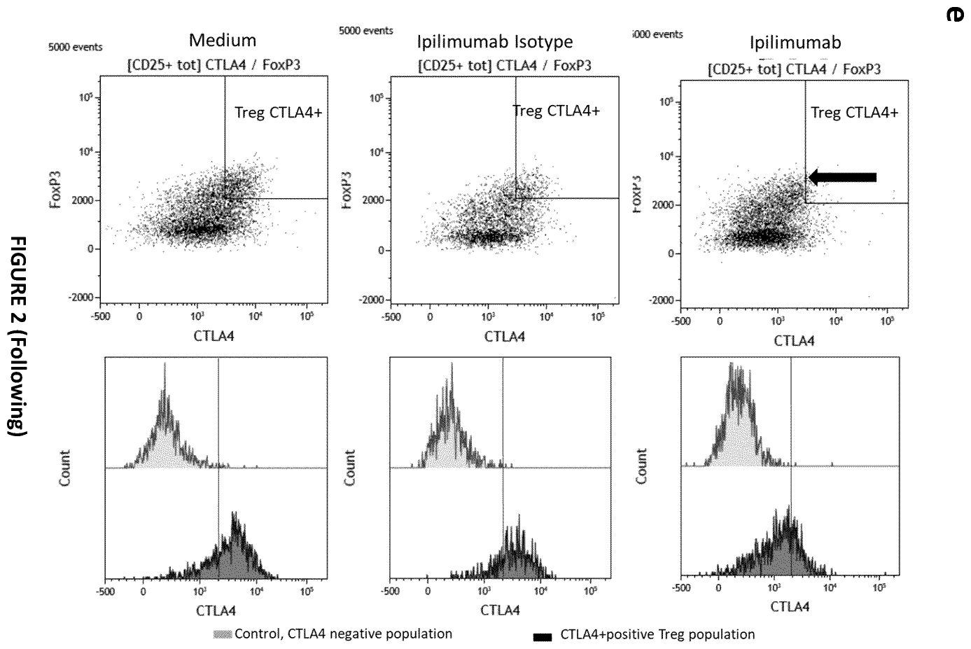

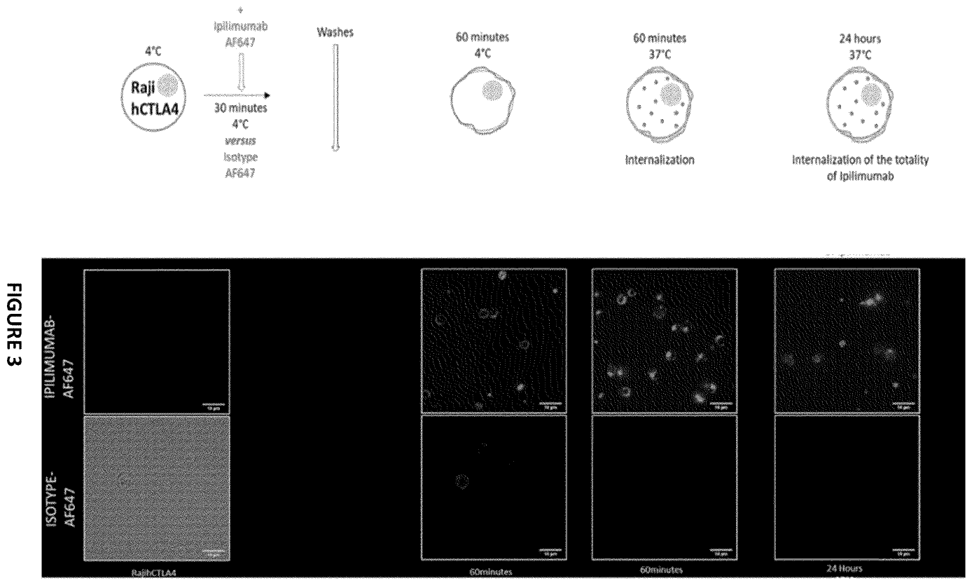

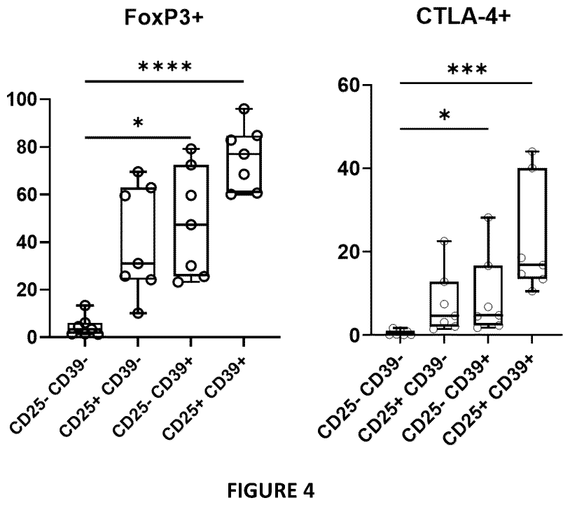

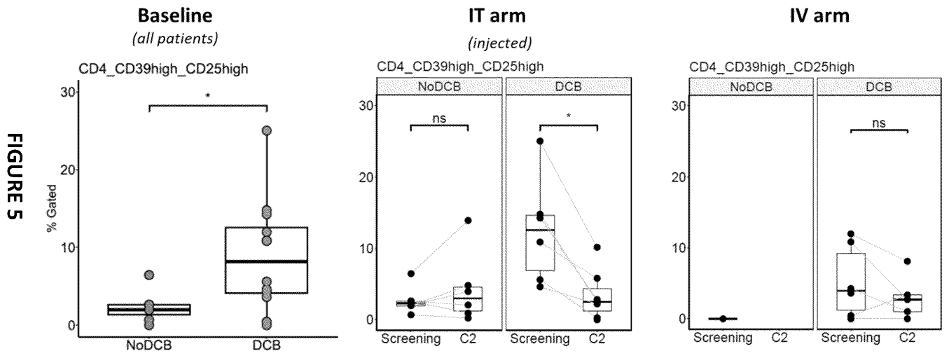

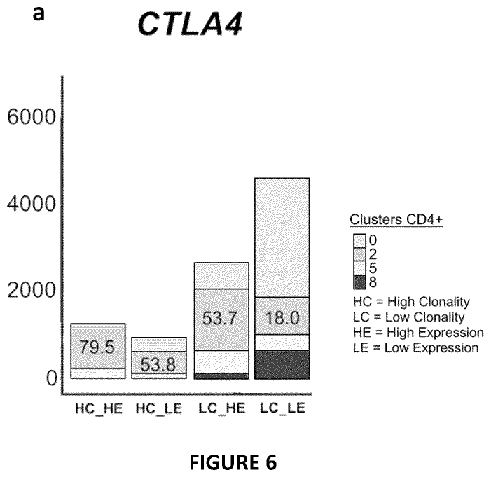

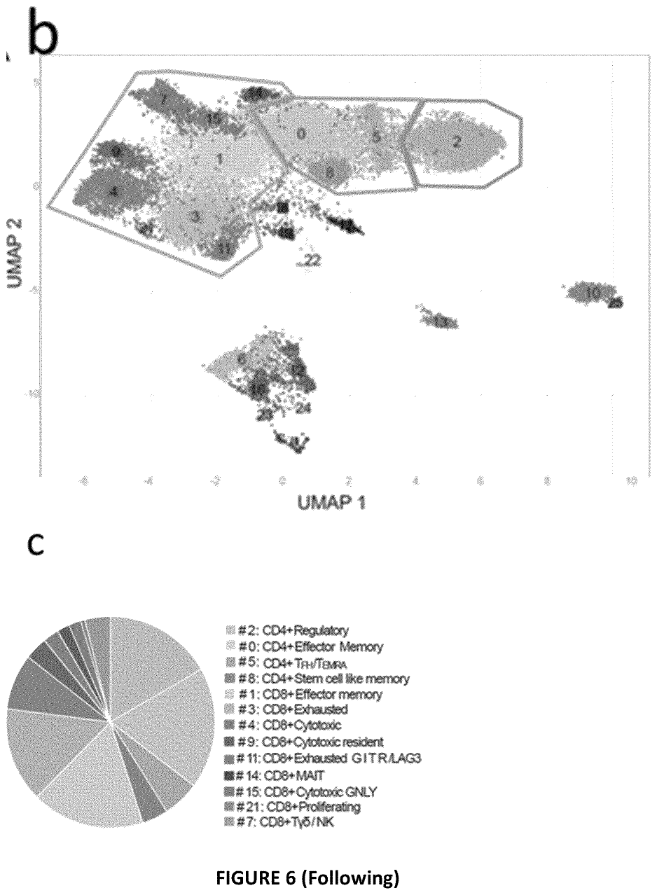

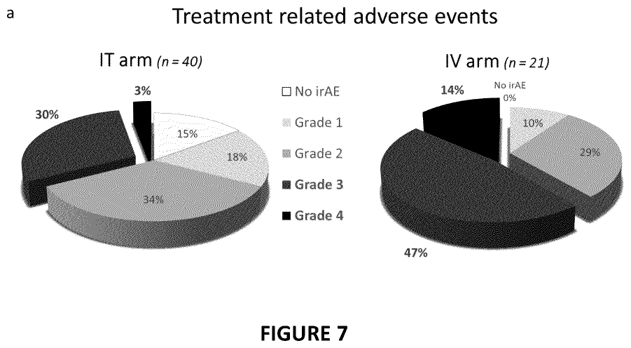

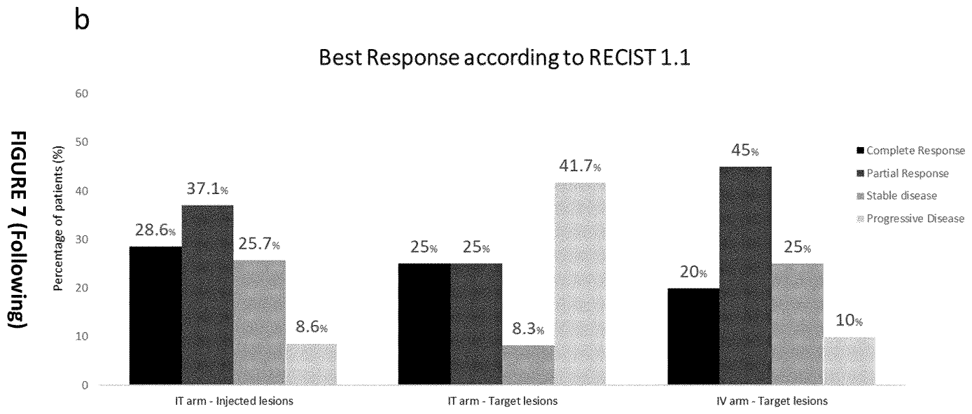

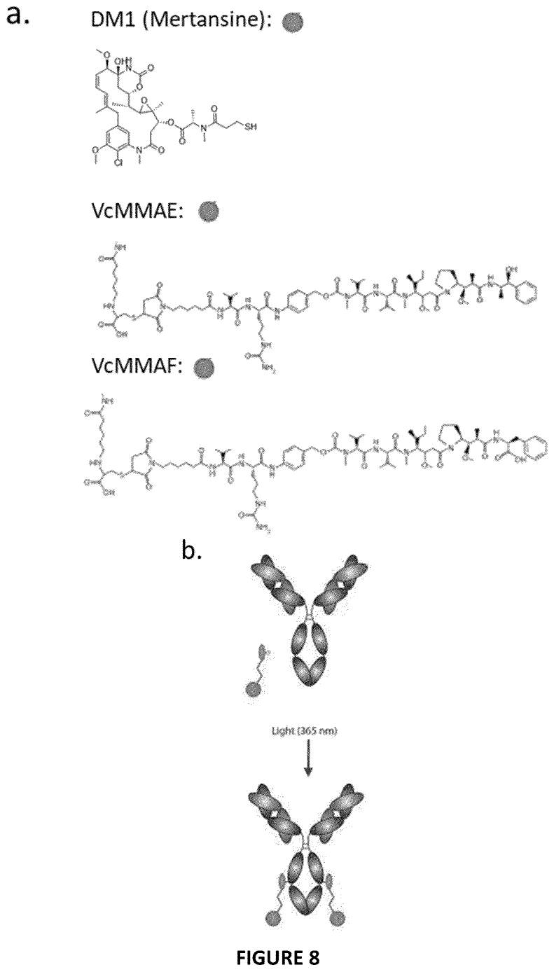

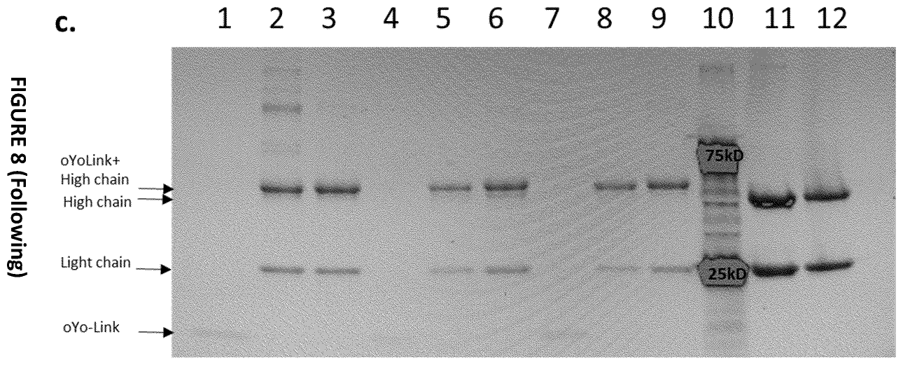

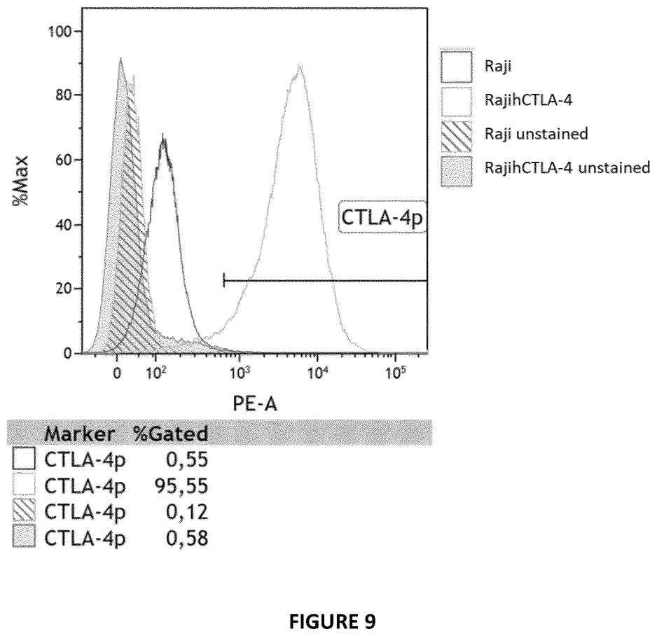

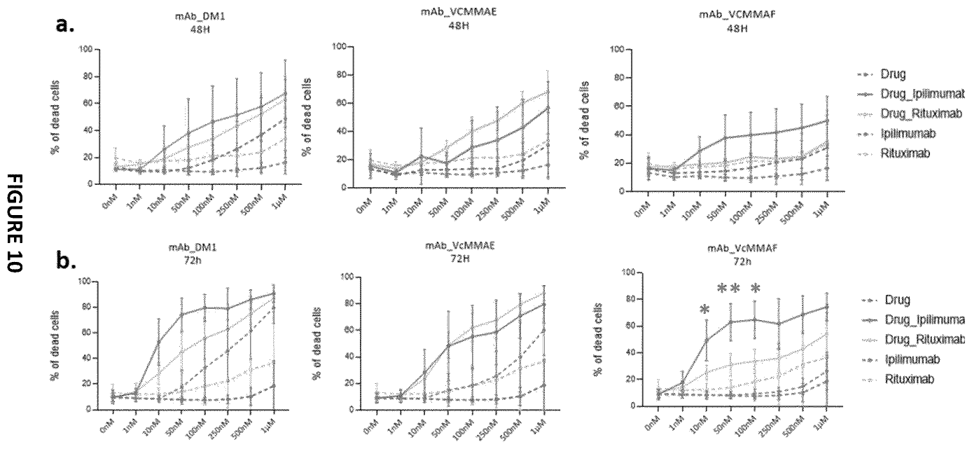

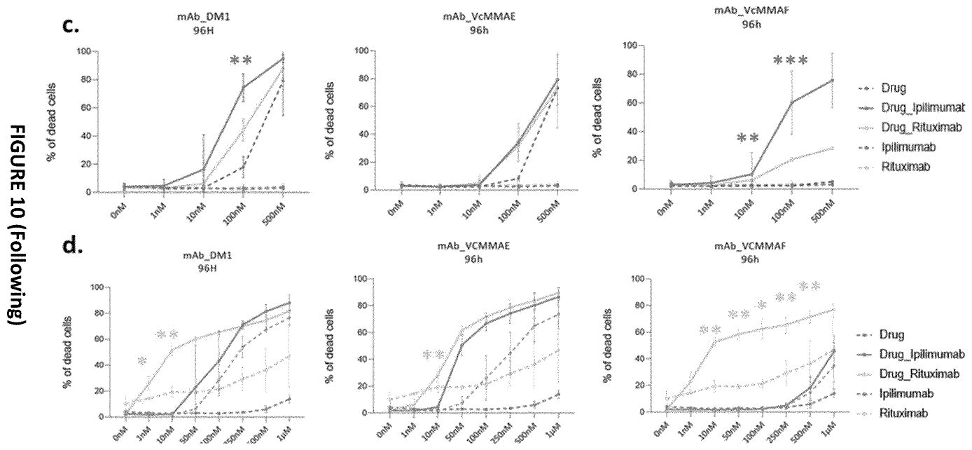

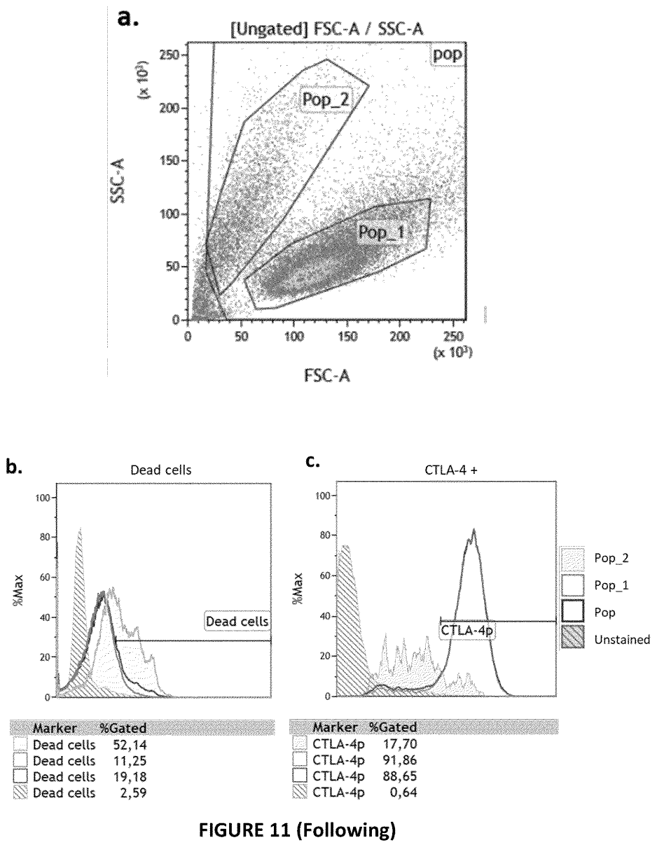

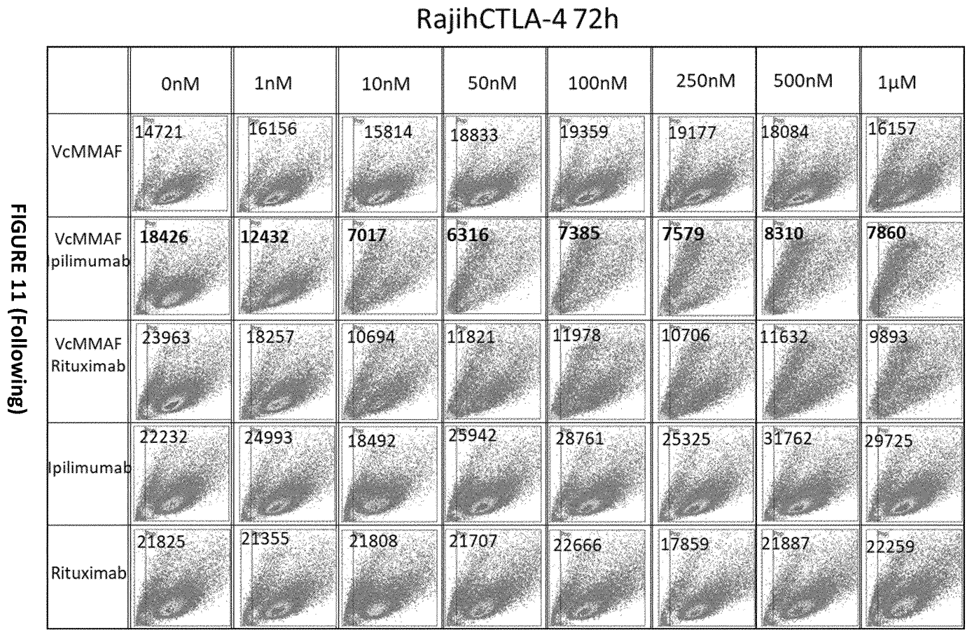

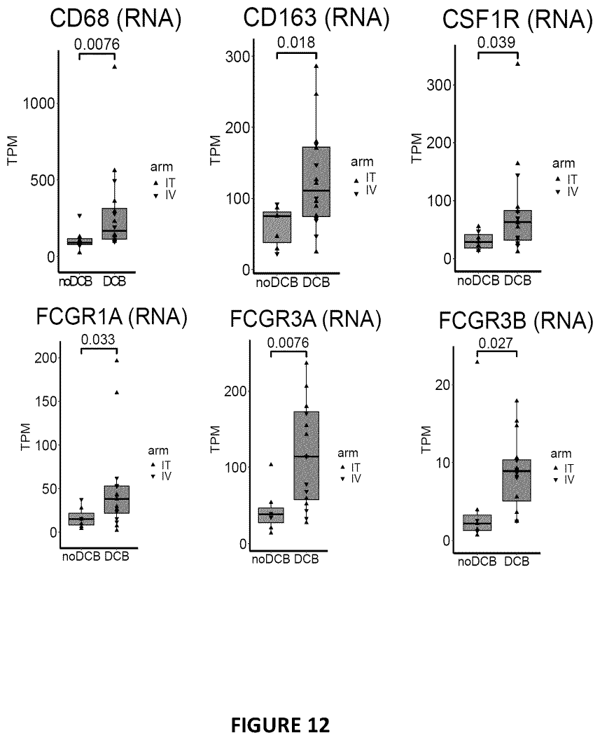

Inventors have recently shown via flow cytometry analysis of freshly resected tumors (FIG. 2) that:
i) CTLA-4 is strongly and predominantly expressed in humans by Tregs in relative proportion to other immune populations and in absolute quantity at the surface of immune cells (Mean Fluorescent Intensity-“MFI”) (FIG. 2a), and
ii) Treg CTLA4+ cells proportion (FIGS. 2c and 2d) decrease upon Ipilimumab treatment, and, for some samples, CTLA4 decrease was stronger than for the majority of the tested samples. Looking at the samples one by one, inventors observed for some of them a shift of this population on the CTLA4 negative side (FIG. 2e).

This observation of lower CTLA4 detection upon ipilimumab exposure allowed inventors to hypothesize that upon fixation to CTLA4, ipilimumab could be internalized together with CTLA4.

They demonstrated that this hypothesis was true by confocal microscopy on a transgenic cell line expressing human CTLA4: after 1 hour of co-incubation of ipilimumab coupled to a fluorochrome together with the CTLA 4+ transgenic cell line at 37 °C, ipilimumab was internalized in membrane intracellular vesicles and after 24 hours at 37 °C, diffuse intracellular staining was observed (FIG. 3). No internalization was observed at 4 °C.

Furthermore, inventors demonstrated on freshly resected and dissociated human tumors that this CD4+FOXP3+CTLA4+ population could be easily detected by double-labelling of CD25+ and CD39+ among CD4+ thus facilitating the detection of Tregs on a single fresh tumor biopsy where it is not possible to perform intra-cytoplasmic FOXP3 labelling due to the low number of cells available (FIG. 4).

In addition, inventors shown in a clinical trial (NIVIPIT trial; NCT02857569) that patients with metastatic melanoma in first line therapy who benefit (DCB patients: Durable Clinical Benefit) from an anti-PD1 (nivolumab) +anti-CTLA4 (ipilimumab) combination are the patients who have a CD4+CD25+CD39+ T cell population in their tumors (FIG. 5; left). This Treg-rich cell population decreases with treatment in patients receiving treatment (DCB). Inventors also showed that this decrease is advantageously increased when the anti-CTLA4 is injected locally in high concentrations (FIG. 5; middle) if compared to the decrease observed when the anti-CTLA4 is injected intravenously (FIG. 5; right).

Inventors also showed by single cell sequencing (single cell RNAseq) of dissociated human tumors that the cell populations expressing high CTLA4 and in clonal expansion are represented at 79.5% by the cluster 2 population (turquoise blue) composed of Tregs (CD4+FOXP3+CD25+CD39+). This clonality of tumour-infiltrating Tregs suggests the Tregs' proliferation after recognition of antigens from the tumour microenvironment (FIG. 6).

In the NIVIPIT clinical trial (NCT02857569), inventors also demonstrated that intra-tumoral injection of small doses of the anti-CTLA4 drug ipilimumab (0.3 mg/kg) significantly reduced the toxicity of the drug while advantageously maintaining a level of efficacy comparable to that obtained at FDA and EMA approved doses in the treatment of metastatic melanoma (FIG. 7). Inventors now herein describe for the first time a drug conjugate comprising a CTLA4 targeting molecule, in particular an intra-tumoral anti-CTLA4 antibody drug conjugate (ADC), offering a very advantageous superior efficacy to IT and IV administered anti-CTLA4 with greatly reduced toxicity, this conjugate being particularly favorable to patients suffering of cancers whose tumors are still in the immuno-editing stage involving CTLA4+ Tregs.

### Anti CTLA4 Antibody Drug Conjugate Construction

As proof of concept, inventors generated 3 ipilimumab antibody drug conjugates with the anti-CTLA4 antibody being conjugated to three different payloads: DM1 (Emtansine), VcMMAE (monomethyl auristatin E) and VcMMAF (monomethyl auristatin F) by using the Alpha Thera's o YoLink® technology. Ipilimumab following the CTLA4 intracellular trafficking (Khailaie et al., 2018), they chose a lysosomally cleavable dipeptide valine-citrulline linker (Alpha Thera's oYoLink® Technology) (FIG. 3). The anti-CD20 Rituximab antibody was coupled to these same drugs as a positive control since the CTLA4+ transgenic cell line was derived from the CD20+ Raji lymphoma cell line.

Inventors validated by SDS gel that the 3 ipilimumab ADC (ipilimumab-DM1, ipilimumab-MMAE and ipilimumab-MMAF) were correctly bound by the valine-citrulline linker to their payload (FIG. 8).

### Ipilimumab-MMAF Anti-CTLA4 Drug Conjugate

To assess the cytotoxicity induced by ipilimumab-DM1, ipilimumab-MMAE and ipilimumab-MMAF, inventors performed in vitro assays on the genetically engineered Raji hCTLA4 cell line (Invivogen) selectively expressing human CTLA4 under Blasticidin selection, and on a Raji control cell line not expressing CTLA4 (FIG. 9).

Different Ipilimumab-ADC concentrations were tested between 0 nM and 1 μM. In order to evaluate the most effective anti-CTLA4 drug conjugate concentrations, they compared the percentage of dead cells induced by the Ipilimumab-Drug conjugates to the Rituximab-Drug conjugates, to Ipilimumab, to Rituximab and to each of the three payloads alone at different time points: 48 hours (FIGS. 10a), 72 hours (FIGS. 10b) and 96 hours (FIG. 10c). They performed an additional control by treating a Raji cell line not expressing CTLA4 during 96 hours with the same experimental conditions (Ipilimumab drug conjugates, Rituximab drug conjugates, Rituximab, Ipilimumab, and payloads alone, at the same concentrations). Cytotoxicity was evaluated by flow cytometry using dead cells labeling (zombie aqua staining). Data suggested that Ipilimumab-MMAF and Ipilimumab-DM1 induced more cytotoxicity after 72 hours (FIGS. 10b) and 96 hours (FIG. 10 c). At 10 nM, Ipilimumab-MMAF induced 50% of dead cells, i.e., as much as Ipilimumab-DM1. At 10 nM, MMAF drug alone induced no cytotoxicity while DM1 drug alone induced 30% of dead cells after a 72 hours-treatment. Treatment of the Raji cell line confirmed that Ipilimumab-MMAF does not induce toxicity in a cell line without CTLA4 expression (FIG. 10d).

### Ipilimumab-MMAF Target Selectively Cells Expressing CTLA4

Forward scatter (FSC) side scatter (SSC) flow cytometry analysis show two populations among the Raji hCTLA4 cell line (FIG. 11a). Some cells lose the CTLA4 transgene resulting in two cell populations. Population 2 which does not express CTLA4 is dying due to blasticidin (FIG. 11b). Raji hCTLA4 phenotyping shows a specific CTLA4 staining of Population 1 (FIG. 11c). By comparing the FSC/SSC of cells treated with different concentrations of Ipilimumab-VcMMAF, Rituximab-VcMMAF, Rituximab, Ipilimumab, and VcMMAF, inventors observed that population 2 is dying under the increasing concentration of Ipilimumab-VcMMAF treatment.

### Conclusion

Inventors showed by confocal microscopy that upon fixation to CTLA4, ipilimumab is internalized together with CTLA4. This observation led them to investigate the possibility of creating a drug conjugate comprising a CTLA4 targeting molecule and to evaluate its efficacy. The experimental results herein described for the first time surprisingly show that Ipilimumab drug conjugates induce more CTLA4+ cell cytotoxicity than the payloads or Ipilimumab alone. Treatment for 72 hours with 10 nm Ipilimumab MMAF induced 50% mortality. Inventors' in vitro experiments show that the effective concentrations of coupled Ipilimumab (10 nM) are 6,6 times lower than the standard concentrations of Ipilimumab treatment. This demonstrate the interest of using such a drug conjugate, for example an anti-CTLA4 drug conjugate such an Ipilimumab-MMAF conjugate, for treating cancer. By targeting tumor specific Treg, the drug conjugate of the invention offers a new and very advantageous therapeutic option, allowing both increased therapeutic efficacy and reduced toxicity for the patient.

### REFERENCES

Antonia, S. J., Villegas, A., Daniel, D., Vicente, D., Murakami, S., Hui, R., Yokoi, T., Chiappori, A., Lee, K. H., de Wit, M., et al. (2017). Durvalumab after Chemoradiotherapy in Stage III Non-Small-Cell Lung Cancer. N. Engl. J. Med. 377, 1919-1929.
Champiat, S. et al. Intratumoral Immunotherapy: From Trial Design to Clinical Practice. Clin. Cancer Res. 27, 665-679 (2021).
Eggermont, A. M. M., Chiarion-Sileni, V., Grob, J. -J., Dummer, R., Wolchok, J. D., Schmidt, H., Hamid, O., Robert, C., Ascierto, P. A., Richards, J. M., et al. (2015). Adjuvant ipilimumab versus placebo after complete resection of high-risk stage III melanoma (EORTC 18071): a randomised, double-blind, phase 3 trial. Lancet. Oncol. 16, 522-530.
Eggermont, A. M. M., Chiarion-Sileni, V., Grob, J. -J., Dummer, R., Wolchok, J. D., Schmidt, H., Hamid, O., Robert, C., Ascierto, P. A., Richards, J. M., et al. (2016). Prolonged Survival in Stage III Melanoma with Ipilimumab Adjuvant Therapy. N. Engl. J. Med. 375, 1845-1855.
Eggermont, A. M. M., Blank, C. U., Mandala, M., Long, G. V., Atkinson, V., Dalle, S., Haydon, A., Lichinitser, M., Khattak, A., Carlino, M. S., et al. (2018). Adjuvant Pembrolizumab versus Placebo in Resected Stage III Melanoma. N. Engl. J. Med. 378, 1789-1801.
Eggermont, A. M. M., Blank, C. U., Mandala, M., and Long, G. V (2020). Longer Follow-Up Con fi rms Recurrence-Free Survival Bene fi t of Adjuvant Pembrolizumab in High-Risk Stage III Melanoma: Updated Results From the EORTC 1325-MG/KEYNOTE-054 Trial abstract. 38.
Ferrara, R., Susini, S., and Marabelle, A. (2019). Anti-CTLA-4 Immunotherapy Does Not Deplete FOXP3 + Regulatory T Cells (Tregs) in Human Cancers—Letter. Clin. Cancer Res. 25, 3468-3468.
Gao, J., Navai, N., Alhalabi, O., Siefker-Radtke, A., Campbell, M. T., Slack Tidwell, R., Guo, C. C., Kamat, A. M., Matin, S. F., Araujo, J. C., et al. (2020) Neoadjuvant PD-L1 plus CTLA-4 blockade in patients with cisplatin-ineligible operable high-risk urothelial carcinoma. Nat. Med.
Khailaie S., Rowshanravan, B., Robert, P. A., Waters E., Halliday, N., Badillo Herrera, J. D., Walker L. S. K., Sansom D. M., Meyer-Hermann M. (2018) Characterization of CTLA4 Trafficking ans Implications for its Function. B. J. 115, 1330-1343 (2018)
Lucca L. E., and Dominguez-Villar, M. (2020). Modulation of regulatory T cell function and stability by co-inhibitory receptors. Nat. Rev. Immunol.
Luke, J. J., Rutkowski, P., Queirolo, P., Del Vecchio, M., Mackiewicz, J., Chiarion-Sileni, V., de la Cruz Merino, L., Khattak, M. A., Schadendorf, D., Long, G. V, et al. (2022). Pembrolizumab versus placebo as adjuvant therapy in completely resected stage IIB or IIC melanoma (KEYNOTE-716): a randomised, double-blind, phase 3 trial. Lancet (London, England) 1-12.
Marabelle, A., Kohrt, H., Sagiv-Barfi, I., Ajami, B., Axtell, R. C., Zhou, G., Rajapaksa, R., Green, M. R., Torchia, J., Brody, J., et al. (2013). Depleting tumor-specific Tregs at a single site eradicates disseminated tumors. J. Clin. Invest. 123, 2447-2463.
Marabelle, A., Tselikas, L., de Baere, T. & Houot, R. Intratumoral immunotherapy: using the tumor as the remedy. Ann. Oncol. 28, xii33-xii43 (2017).
Marabelle, A. et al. Starting the fight in the tumor: expert recommendations for the development of human intratumoral immunotherapy (HIT-IT). Ann. Oncol. Off. J. Eur. Soc. Med. Oncol. 29, 2163-2174-(2018).
Plitas, G., and Rudensky, A. Y. (2020). Regulatory T Cells in Cancer. Annu. Rev. Cancer Biol. 4, 459-477.
Rowshanravan, B., Halliday, N., and Sansom, D. M. (2018). CTLA-4: A moving target in immunotherapy. Blood 131, 58-67.
Selby, M. J., Engelhardt, J. J., Quigley, M., Henning, K. A., Chen, T., Srinivasan, M., and Korman, A. J. (2013). Anti-CTLA-4 Antibodies of IgG2a Isotype Enhance Antitumor Activity through Reduction of Intratumoral Regulatory T Cells. Cancer Immunol. Res. 1, 32-42.
Sharma, A., Subudhi, S. K., Blando, J., Scutti, J., Vence, L., Wargo, J., Allison, J. P., Ribas, A., and Sharma, P. (2019). Anti-CTLA-4 immunotherapy does not deplete Foxp3 p regulatory T cells (Tregs) in human cancers. Clin. Cancer Res. 25, 1233-1238.
Simpson, T. R., Li, F., Montalvo-Ortiz, W., Sepulveda, M. A., Bergerhoff, K., Arce, F., Roddie, C., Henry, J. Y., Yagita, H., Wolchok, J.D., et al. (2013). Fc-dependent depletion of tumor-infiltrating regulatory T cells co-defines the efficacy of anti-CTLA-4 therapy against melanoma. J. Exp. Med. 210, 1695-1710.
Tay, C., Tanaka, A., and Sakaguchi, S. (2023). Tumor-infiltrating regulatory T cells as targets of cancer immunotherapy. Cancer Cell 41, 450-465.
Tselikas, L. et al. Interventional radiology for local immunotherapy in oncology. Clin. Cancer Res. clincanres. 4073.2020(2021 ) doi:10.1158/1078-0432.ccr-19-4073.
Tselikas, L. et al. Interventional radiology for local immunotherapy in oncology. Clin. Cancer Res. clincanres. 4073.2020(2021 ) doi:10.1158/1078-0432.ccr-19-4073.
Tselikas, L., et al. (2020). Pickering emulsions with ethiodized oil and nanoparticles for slow release of intratumoral anti-CTLA4 immune checkpoint antibodies. J. Immunother. Cancer 8, e000579.

1-18. (canceled)

19. A drug conjugate comprising i) an anti-cytotoxic-T-lymphocyte-antigen 4 protein (anti-CTLA4) monoclonal antibody, ii) at least one cytotoxic agent, and iii) a linker connecting the CTLA4 targeting molecule and the cytotoxic agent.

20. The drug conjugate according to claim 19, wherein the anti-CTLA4 monoclonal antibody is ipilimumab or tremelimumab.

21. The drug conjugate according to claim 19, wherein the linker is a cleavable linker.

22. The drug conjugate according to claim 21, wherein the cleavable linker is an acid-labile linker, an enzyme-cleavable linker, a lysosomal protease-sensitive linker, a disulfide linker or a β-glucuronide linker.

23. The drug conjugate according to claim 19, wherein the cytotoxic agent is selected from the group consisting of a microtubule inhibitor, a DNA damaging agent, an alkylating agent, a platinum complex, a cytotoxic antibiotic, an antimetabolite, a topoisomerase inhibitor, a RNA polymerase inhibitor, an antimitotic agent, and a combination thereof.

24. The drug conjugate according to claim 23, wherein the microtubule inhibitor is selected from emtansine (DM1), auristatin and a derivative thereof selected from the group consisting of monomethyl auristatin E (MMAE) and monomethyl auristatin F (MMAF).

25. The drug conjugate according to claim 20, wherein the conjugate comprises i) ipilimumab, ii) a cytotoxic agent, and iii) a cleavable linker comprising a valine-citrulline dipeptide or a phenylalanine-lysine dipeptide.

26. A pharmaceutical composition comprising a pharmaceutically acceptable support and at least one drug conjugate according to claim 19 and:
a) a second conjugate comprising a distinct cytotoxic agent; or
b) distinct therapeutic agent(s) selected from an immune checkpoint targeting agent, an anti-angiogenic agent, a cytotoxic agent and an hormonal agent, and a pharmaceutically acceptable support, excipient, diluent, or carrier.

27. A kit comprising:
a) at least two drug conjugates according to claim 19, said first drug conjugate comprising a first cytotoxic agent and said second drug conjugate comprising a second cytotoxic agent; or
b) a drug conjugate comprising i) an anti-cytotoxic-T-lymphocyte-antigen 4 protein (anti-CTLA4) monoclonal antibody, ii) at least one cytotoxic agent, and iii) a linker connecting the CTLA4 targeting molecule and the cytotoxic agent and distinct therapeutic agent(s) selected from the group consisting of an immune checkpoint targeting agent, an anti-angiogenic agent, a cytotoxic agent, an hormonal agent and any combination thereof.

28. A method of treating cancer or preventing relapse of cancer comprising administering the drug conjugate according to claim 19 or a pharmaceutical composition comprising said drug conjugate to a subject having cancer or at risk of cancer relapse.

29. The method according to claim 28, wherein the cancer is a solid cancer selected from melanoma, renal cell carcinoma (RCC), colorectal cancer, hepatocellular carcinoma, non-small cell lung cancer (NSCLC) and mesothelioma (MM), or a hematological malignancy selected from a lymphoma and a leukemia.

30. The method according to claim 28, wherein the cancer cells or the cancer tumor microenvironment (TME) comprise(s) CTLA4 expressing cells.

31. The method according to claim 30, wherein the CTLA4 expressing cells are regulatory T-cells.

32. The method according to claim 28, wherein the drug conjugate or composition is administered to the subject as a neoadjuvant therapeutic agent, before any partial or total tumor surgical resection or focal destruction by any cytoreductive strategy, in monotherapy or in combination with distinct anti-cancer therapeutic agent(s).

33. The method according to claim 28, wherein about 0.01 mg to about 1 mg of the drug conjugate is administered to the subject per kg body weight of said subject.

34. The method according to claim 28, wherein the subject is a non-human mammal or a human being.

35. The method according to claim 28, wherein the subject is a subject whose tumor or Tumor Micro Environment (TME) comprises CTLA4+ tumor cells and/or CTLA4+ immune cells.

36. The method according to claim 35, wherein the subject has resistance to naked anti-CTLA4 and/or anti-PD(L)1 agent(s).

37. The method according to claim 35, wherein the subject is a subject whose tumor and/or TME display(s) no, low level of, or dysfunctional myeloid cells, said myeloid cells expressing no or low levels of the Fcγ receptor I (“CD64”), Fcγ receptor IIIa (“CD16a”) and/or Fcγ receptor IIIb (“CD16b”).

38. The method according to claim 31, wherein the regulatory T-cells are CD4+FOXP3+CTLA4+ T cells and/or CD4+CD25+CD39+ T cells which comprise tumor antigen-specific clonally expanding Treg cells.
